SEQUENCE LISTING (35 sequences)

SEQ ID NO: 1 - protein, 613 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 2 - protein, 744 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 3 - protein, 705 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 4 - protein, 651 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSGGSGSDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 5 - protein, 739 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 6 - protein, 715 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 7 - protein, 515 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFL

SEQ ID NO: 8 - protein, 621 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 9 - protein, 594 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 10 - protein, 640 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 11 - protein, 25 aa, synthetic construct
MELLILKANAITTILTAVTFCFASG

SEQ ID NO: 12 - protein, 25 aa, synthetic construct
MELLILKANVIATILTAVTFCFASS

SEQ ID NO: 13 - protein, 18 aa, synthetic construct
MSWKVVIIFSLLITPQHG

SEQ ID NO: 14 - protein, 488 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 15 - protein, 15 aa, synthetic construct
GSGGSGSGSGGSGSG

SEQ ID NO: 16 - protein, 12 aa, synthetic construct
GSGGSGSGSGGS

SEQ ID NO: 17 - protein, 8 aa, synthetic construct
GGSGGSGS

SEQ ID NO: 18 - protein, 8 aa, synthetic construct
GSGGSGSG

SEQ ID NO: 19 - protein, 11 aa, synthetic construct
EKAAKAEEAAR

SEQ ID NO: 20 - protein, 12 aa, synthetic construct
EKAAKAEEAARK

SEQ ID NO: 21 - protein, 27 aa, synthetic construct
GYIPEAPRDGQAYVRKDGEWVLLSTFL

SEQ ID NO: 22 - protein, 157 aa, synthetic construct
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 23 - protein, 157 aa, synthetic construct
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 24 - protein, 203 aa, synthetic construct
MEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 25 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 26 - protein, 157 aa, synthetic construct
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 27 - protein, 122 aa, synthetic construct
MVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 28 - protein, 157 aa, synthetic construct
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 29 - protein, 103 aa, synthetic construct
MEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 30 - protein, 203 aa, synthetic construct
MEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 31 - protein, 119 aa, synthetic construct
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 32 - protein, 153 aa, synthetic construct
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 33 - skipped

SEQ ID NO: 34 - protein, 488 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 35 - protein, 488 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

(19) United States
(12) Patent Application Publication
(10) Pub. No.: US 2024/0350616 A1
(43) Pub. Date: Oct. 24, 2024

(54) VIRUS-LIKE PARTICLE VACCINE FOR RESPIRATORY SYNCYTIAL VIRUS

(71) Applicant: Icosavax, Inc., Seattle, WA (US)

(72) Inventors: Niranjan Kanesa-Thasan, Lexington, MA (US); Douglas Holtzman, Seattle, WA (US)

(21) Appl. No.: 18/682,281

(22) PCT Filed: Aug. 9, 2022
(86) PCT No.: PCT/US22/74699

§ 371 Date: Feb. 8, 2024

Related U.S. Application Data
(60) Provisional application No. 63/367,103, filed on Jun. 27, 2022.
(60) Provisional application No. 63/231,568, filed on Aug. 10, 2021.

(51) Int. Cl. A61K 39/155; A61K 39/00; A61P 31/14; C12N 7/00

(52) U.S. Cl. CPC A61K 39/155; A61P 31/14; C12N 7/00; A61K 2039/5258; A61K 2039/545; A61K 2039/55; A61K 2039/55505; A61K 2039/55566; A61K 2039/575; A61K 2039/70; C12N 2760/18522; C12N 2760/18523; C12N 2760/18534

(57) ABSTRACT

The present disclosure relates to targeting Respiratory Syncytial Virus (RSV), and methods of using such vaccines to treat infections with RSV, in particular, lower respiratory tract infections (LRTIs).

### RELATED APPLICATIONS

This application claims the benefit of priority under 35 U.S.C. § 119(e) to U.S. Provisional Application No. 63/367,103, filed Jun. 27, 2022, and to U.S. Provisional Application No. 63/231,568, filed Aug. 10, 2021, each of which is incorporated herein by reference in its entirety.

### INCORPORATION BY REFERENCE OF SEQUENCE LISTING

This application contains a Sequence Listing which has been submitted in ASCII format via EFS-WEB and is hereby incorporated by reference in its entirety. Said ASCII copy, created on Aug. 8, 2022 is named 061291-506001WO_ST26.xml and is 40 kilobytes in size.

### FIELD OF THE DISCLOSURE

The present disclosure relates to vaccines for Respiratory Syncytial Virus (RSV).

### BACKGROUND

Respiratory syncytial virus is a single-strand, negative sense RNA virus of the family Pneumoviridae. RSV circulates seasonally and is a major cause of lower respiratory tract infection (LRTI) worldwide. Epidemiological data suggest that in the US alone RSV may cause >170,000 hospitalizations and ˜14,000 deaths annually (Colosia et al., PLoS One. 2017; 12(8):e0182321). Similarly, RSV is an important cause of respiratory disease in Europe (Broberg et al., Euro Surveill. 2018; 23(5):17-00284).

Accumulating data has identified a substantial disease burden in adults, comparable to influenza, with most of the hospitalization and mortality occurring in older adults over 60 years of age (Falsey et al., J Infect Dis. 2014; 209(12):1873-81; Fleming et al. BMC Infect Dis. 2015; 15(1):443). The incidence and severity of RSV disease is particularly high in the frail elderly and in older adults with cardiopulmonary conditions, who are considered at high risk for complications and hospitalization (Falsey et al., N Engl J Med. 2005; 352(17):1749-59). In addition to the burden of disease in older adults, RSV infections are a major cause of bronchiolitis and pneumonia in young children globally (Nair et al., Lancet. 2010; 375(9725):1545-55). A large proportion of these are lower respiratory tract infection (LRTI) in the first twelve months of life, such that RSV is the single most important viral LRTI during infancy and early childhood worldwide, and particularly in preterm infants and in infants with cardiopulmonary conditions, who are considered at high risk for complications and hospitalization. Data from the US suggest that RSV is one of the leading causes of acute respiratory illness (ARI) in infants, with an estimated 2 million cases annually (Lee et al., Hum Vaccin. 2005; 1(1):6-11).

Treatment for illness caused by RSV (e.g., RSV-A and/or RSV-B) is mainly symptomatic and prevention consists mainly of infection control strategies, such as hand washing and droplet precautions. Neonates at high risk for RSV infection, such as premature infants or infants with cardiopulmonary disease, are candidates for prophylaxis with humanized monoclonal antibody palivizumab (Resch et al., Hum Vaccin Immunother. 2017; 13(9):2138-2149), which has been shown to be only moderately effective (45-55%) in reducing hospitalization for RSV (Ambrose et al., Human Vaccines & Immunotherapeutics. 2014; 10:10, 2785-2788). Antiviral agents for the prevention and treatment of RSV infections in elderly adults are currently not available, and there is no vaccine licensed for prevention of disease due to RSV infections.

RSV F protein is a major conserved surface antigen of RSV and antibodies against it are associated with protection against disease. RSV F-protein is a validated target for protection against infection by RSV as demonstrated by the clinical efficacy of palivizumab, a monoclonal antibody that binds F-antigen and leads to neutralization of the virus (Johnson et al., J Infect Dis. 1997 November; 176(5):1215-24). RSV-F protein is known to undergo a significant change in structure from prefusion to postfusion form which catalyzes viral and host membrane fusion to allow for viral entry into the cell (McLellan et al., Science. 2013; 342(6158):592-8). Prefusion F-protein has important epitopes that are lost during the transition to postfusion F-protein (Melero et al., Vaccine. 2017; 35(3):461-468). Antibody depletion studies with human sera absorbed with RSV F protein in either conformation demonstrate that the majority of the neutralizing response against RSV F protein targets the prefusion structure (Krarup et al., Nat Commun. 2015; 6:8143). These studies also demonstrate the potential for antibodies that bind postfusion F protein to interfere with neutralization (Ngwuta et al., Sci Transl Med. 2015; 7(309):309ra162). In general, high levels of antibodies against RSV F protein are associated with protection against severe disease. However, generating high-titers of neutralizing antibodies against RSV F protein remains challenging, due to the specific biochemical nature of the RSV F protein and the unpredictability of vaccine responses to RSV F.

There is thus a need for novel vaccines targeting RSV (e.g., RSV-A subtype and/or RSV-B subtype) to induce high neutralizing antibody levels. The compositions and methods of the present disclosure address that need.

### BRIEF SUMMARY

In one aspect, provided herein is a pharmaceutical composition, comprising a protein complex comprising a first component comprising an RSV F protein and a first multimerization domain, and a second component comprising a second multimerization domain; and one or more pharmaceutically acceptable diluents or excipients. In some embodiments, the pharmaceutical composition comprises an oil-in-water adjuvant. In some embodiments, the pharmaceutical composition comprises an aluminum hydroxide-adjuvant. In some embodiments, the protein complex is an icosahedral protein complex. In some embodiments, the protein complex comprises 20 copies of the first component and 12 copies of the second component.

In some embodiments, the RSV F protein comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 14, 34, and 35. In some embodiments, the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 24 and 30-31; and/or the second multimerization domain comprises an amino acid sequence which is at least 95%, at least 96%, at least 97%, at least 98%, at least 99%, or at least 100% identical to an amino acid sequence selected from any one of SEQ ID NOS: 22-23, 25-29, and 32. In some embodiments, the first component comprises an amino acid sequence which is at least 75%, 80%, 85%, 90%, 91%, 92%, 93%, 94%, 95%, 96%, 97%, 98%, 99%, or 100% identical to SEQ ID NO: 6; and the second component comprises an amino acid sequence which is at least 75%, 80%, 85%, 90%, 91%, 92%, 93%, 94%, 95%, 96%, 97%, 98%, 99%, or 100% identical to SEQ ID NO: 26.

In another aspect, provided herein is a unit dose of the pharmaceutical composition comprising about 0.5 μg to about 1 μg, about 20 μg to about 25 μg, about 70 μg to about 75 μg, about 100 μg to about 125 μg, or about 200 μg to about 250 μg of the protein complex.

In another aspect, provided herein is a method of vaccinating a subject, comprising administering to the subject an effective amount of a pharmaceutical composition provided herein. In another aspect, provided herein is a method of generating an immune response in a subject, comprising administering to the subject an effective amount of a pharmaceutical composition provided herein. In another aspect, provided herein is a method of preventing RSV disease in a subject, comprising administering to the subject an effective amount of a pharmaceutical composition provided herein. In some embodiments, the subject is at risk of severe RSV disease. In some embodiments, the subject is an adult of over 60 years of age. In some embodiments, the subject is a healthy adult of 18-45 years of age.

In another aspect, provided herein is a method of generating an immune response in an unborn child, the method comprising administering an effective amount of a pharmaceutical composition provided herein to the mother of said unborn child. In some embodiments, the pharmaceutical composition is administered to the mother in the last trimester of the pregnancy.

In some embodiments, an effective amount of a pharmaceutical composition comprises about 0.5 μg to about 1 μg, about 20 μg to about 25 μg, about 70 μg to about 75 μg, about 100 μg to about 125 μg, or about 200 μg to about 250 μg of the protein complex.

In some embodiments, a method provided herein further comprising administering a second dose of a pharmaceutical composition provided herein. In some embodiments, the second dose is administered within about 2, 3, 4, 5, 6, 7, 8, 9, 10, 11, or 12 weeks, about 1 month, about 2 months, about 3 months, about 4 months, about 5 months, about 6 months, about 9 months, or about 12 months of the first dose. In some embodiments, the method provided herein further comprises administering a third dose of a pharmaceutical composition provided herein. In some embodiments, the third dose is administered about 1 year, about 2 years, about 3 years, about 4 years, or about 5 years after the second dose. In some embodiments, a method provided herein further comprises administering subsequent doses at regular intervals of about 1, 2, 3, 4 or 5 years.

In some embodiments, a method provided herein limits the development of an RSV infection in a subject. In some embodiments, the method results in the production of RSV-A-specific neutralizing antibodies in the subject. In some embodiments, the method results in an increase in RSV-A-specific neutralizing antibodies in the subject of at least about 3-fold, at least about 4-fold, at least about 5-fold, at least about 10-fold, at least about 15-fold, at least about 20-fold, or at least about 25-fold compared to baseline. In some embodiments, the increase in RSV-A-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition. In some embodiments, the method results in the production of RSV-B-specific neutralizing antibodies in the subject. In some embodiments, the method results in an increase in RSV-B-specific neutralizing antibodies in the subject of at least about 2-fold, at least about 3-fold, at least about 4-fold, at least about 5-fold, at least about 10-fold, at least about 15-fold, at least about 20-fold, or at least about 25-fold compared to baseline. In some embodiments, the increase in RSV-B-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of RSV F-protein-specific IgG antibodies in the subject. In some embodiments, the method results in an increase of RSV F-protein-specific neutralizing antibodies in the subject of at least about 3-fold, at least about 4-fold, at least about 5-fold, at least about 10-fold, at least about 15-fold, at least about 20-fold, or at least about 25-fold compared to baseline. In some embodiments, the increase in RSV F-protein-specific IgG antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of RSV F-protein-specific memory-B-cells in the subject. In some embodiments, the method results in an increase in RSV F-protein-specific memory-B-cells in the subject of at least about 3-fold, at least about 4-fold, at least about 5-fold, at least about 10-fold, at least about 15-fold, at least about 20-fold, or at least about 25-fold compared to baseline. In some embodiments, the increase in RSV F-protein-specific memory-B-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

In some embodiments, the method results in the production of RSV F-protein-specific T-cells in the subject. In some embodiments, the method results in an increase in RSV F-protein-specific T-cells in the subject of at least about 3-fold, at least about 4-fold, at least about 5-fold, at least about 10-fold, at least about 15-fold, at least about 20-fold, or at least about 25-fold compared to baseline. In some embodiments, the increase in RSV F-protein-specific T-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

### BRIEF DESCRIPTION OF THE SEVERAL VIEWS OF THE DRAWINGS

FIGS. 1A-1C shows neutralization antibody titers measured in naïve mice on Days 0 (FIG. 1A), 42 (FIG. 1B) and 56 (FIG. 1C) after administration of RSV vaccine (two-tailed, unpaired t test). Group 1: RSV vaccine (8.33 μg); Group 2: RSV vaccine (8.33 μg) plus Alhydrogel; Group 3: RSV vaccine (2.5 μg); Group 4: RSV vaccine (2.5 μg) plus Alhydrogel; Group 5: RSV vaccine (0.83 μg); Group 6: RSV vaccine (0.83 μg) plus Alhydrogel; Group 7: RSV vaccine (8.33 μg) plus Addavax; Group 8: control sera.

FIG. 2 shows the increase in neutralizing antibody titers with administration of Alhydrogel-adjuvanted or non-adjuvanted RSV vaccine or RSV F-protein in RSV-primed mice.

FIG. 3 shows RSV neutralizing antibody titers in rabbits; impact of pre-immunization with VLP core. Group 1=5 New Zealand White (NZW) female rabbits were administered with VLP core plus Addavax (oil-in-water emulsion) on Days 1 and 14. Subsequently, the rabbits were vaccinated with RsV vaccine plus Alhydrogel (aluminum hydroxide adjuvant) on Days 56, 70 and 84. Group 2=NZW 3 female rabbits each were vaccinated with RSV vaccine without Alhydrogel and no VLP core prior administration. Group 3=NZW 3 female rabbits each were vaccinated with RSV vaccine with Alhydrogel and no VLP core prior administration.

FIG. 4 is the study design for a Phase 1/1b randomized, observer-blinded, placebo-controlled study to evaluate IVX-121 administration in young and older adult subjects.

FIG. 5 shows a summary of safety data. There were no serious adverse events (SAEs), AEs of special interest (AESIs) or adverse events (AEs) leading to study withdrawal.

FIG. 6 shows graphs of solicited systemic adverse events within 7 days of a single dose, maximal severity (“alum”=500 μg/mL aluminum hydroxide). Unadjuvanted IVX-121 reactogenicity is mild in older adults with similar tolerability to placebo.

FIG. 7 shows a graph of RSV-A neutralizing antibodies (nAB). Geometric mean titer (GMT) is expressed in international units per milliliter (IU/mL). GMT of unadjuvanted IVX-121 are comparable in young and older adults.

FIG. 8 shows a graph of RSV-A neutralizing antibodies (nAB), unadjuvanted versus adjuvanted. Geometric mean titer (GMT) is expressed in international units per milliliter (IU/mL). Alum adjuvant had no beneficial effect in young and older adults.

FIG. 9 shows tables summarizing neutralizing and binding antibody data.

### DETAILED DESCRIPTION

Provided herein are pharmaceutical compositions comprising a protein complex which may be used to vaccinate against RSV (e.g., RSV-A subtype and/or RSV-B subtype). In particular, provided herein is a composition comprising a non-replicating recombinant protein-based vaccine which is presented to the immune system as a virus-like particle (VLP). Several naturally occurring VLPs are constituents of licensed vaccines (e.g., hepatitis B, human papilloma virus) and have been safely used across all age groups, ranging from young children to older adults. Without wishing to be bound by theory, it is thought that such a vaccine can boost RSV-neutralizing antibody titers while reducing the induction of binding, non-neutralizing antibodies which were previously associated with enhanced respiratory disease (ERD).

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless otherwise specified, the term “sequence identity” refers to sequence identity calculated as percentage of exact matches between the reference sequence and the sequence of interest, across the entire length of the reference sequence, when the sequence of interest is aligned to the reference sequence using the Blast-p program of the National Center for Biotechnology Information (NCBI) online alignment tool, version 2.11.0 (released Oct. 19, 2020), Altschul et al. J. Mol. Biol. 215:403-410 (1990).

As used herein, the terms “heterologous vaccine” and “heterologous vaccination” refer to a vaccine given to a subject who has received or will receive a vaccination for the same indication (e.g., RSV) using a vaccine made with another technology (e.g., an mRNA vaccine, adenoviral vector vaccine, or a protein subunit vaccine). As such, a “heterologous vaccine” refers to a vaccine made using a different technology type than the reference vaccine.

A “heterologous boost” or “heterologous boost vaccine” refers to a heterologous vaccine (e.g., a protein-based VLPs) given to a subject who has received a vaccination for the same indication (e.g., RSV) using a vaccine made with another technology (e.g., an mRNA vaccine, adenoviral vector vaccine, or a protein subunit vaccine).

The term “prime vaccine” refers to the first vaccine in a vaccination protocol or to a first set of vaccines administered prior to a heterologous boost vaccine. For example, an mRNA vaccine or adenoviral vaccine may be administered first, optionally followed by a second prime vaccine after a suitable interval, and then the heterologous vaccine may be administered. The heterologous vaccine may serve to “boost” the immune response to the prime vaccine. A “priming vaccine” as used herein refers to a vaccine comprising an agent(s) that encodes the target antigen to which an immune response is to be generated. Priming vaccines are administered to the subject in an amount effective to elicit an immune response to the target antigen.

A “heterologous prime-boost vaccination” refers to a vaccine given to a subject who will receive a vaccination for the same indication (e.g., RSV) using a vaccine made with another technology. For example, the initial dose (primary vaccine or prime vaccination) of a vaccine may an mRNA vaccine (or alternatively, the subject may have been diagnosed with the indication, e.g., RSV), and subsequently receive a second vaccination for the same indication, wherein the second vaccination is of a different technology—a heterologous vaccination (e.g., a protein-based VLP). In examples, heterologous prime-boost vaccination includes a primary vaccination for an indication, and a subsequent vaccination for the same indication, wherein the heterologous vaccination is administered 3 months to 6 months after the heterologous prime vaccine, or 4 or more months after a heterologous prime vaccine, or 6 months or more after a heterologous prime vaccine, or 10 months or more after a heterologous prime vaccine. In yet other examples, the heterologous boost vaccination is administered 1 year after a heterologous prime vaccine. A “heterologous prime” or “heterologous prime vaccine” refers to a vaccine given to a subject who will receive a vaccination for the same indication (e.g., RSV) using a vaccine made with another technology (e.g., an mRNA vaccine, adenoviral vector vaccine, or a protein subunit vaccine).

The term “virus-like particle” or “VLP” refers to a molecular assembly that resembles a virus, but is non-infectious, and that displays an antigenic protein, or antigenic fragment thereof, of a viral protein or glycoprotein. A “protein-based VLP” refers to a VLP formed from proteins or glycoproteins and substantially free of other components (e.g., lipids). Protein-based VLPs may include post-translation modification and chemical modification, but are to be distinguished from micellar VLPs and VLPs formed by extraction of viral proteins from live or live inactivated virus preparations. The term “designed VLP” refers to a VLP comprising one or more polypeptides generated by computational protein design. The term “symmetric VLP” refers to a protein-based VLP with a symmetric core. These include but are not limited to designed VLPs. For example, the protein ferritin has been used to generate a symmetric, protein-based VLP using naturally occurring ferritin sequences. Ferritin-based VLP are distinguished from designed VLPs in that no protein engineering is necessary to form a symmetric VLP from ferritin, other than fusing the viral protein to the ferritin molecule. Protein design methods can be used to generate similar one- and two-component nanostructures based on template structures (e.g., structures deposited in the Protein Data Bank) or de novo (i.e., by computational design of new proteins having a desired structure but little or no homology to naturally occurring proteins). Such one- and two-component nanostructures can then be used as the core of a designed VLP. The terms “protein nanoparticle” or “nanoparticle” and the term “nanostructure” may be used to refer to protein-based VLPs as described herein.

As used herein, an “immunogenic composition” is a composition that comprises an antigen where administration of the composition to a subject results in the development in the subject of a humoral and/or a cellular immune response to the antigen.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular, aspects, the subject is up to 4 months old, or up to 6 months old. In some embodiments, the adults are older adults, such as older adults over 50 years of age, older adults over 55 years of age, older adults over 60 years of age, older adults over 65 years of age. In some embodiments, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

### Protein Complexes

The present disclosure relates generally to vaccination of a subject with a protein complex comprising a first component comprising an RSV F protein and a first multimerization domain. The protein complex may comprise the F protein of RSV-A or RSV-B. Illustrative sequences of the F proteins of RSV-A and B are set forth in SEQ ID NOs: 14 and 34, respectively. The F protein portion and the first multimerization domain may be linked by any suitable means, including co-expression as a fusion protein. The protein complex may optionally comprise a second component comprising a second multimerization domain. The pharmaceutical composition typically comprises one or more pharmaceutically acceptable diluents or excipients.

In some embodiments, the protein complex is a nanostructure, nanoparticle, or protein-based virus-like particle.

In some embodiments, the protein complex is an icosahedral protein complex, such as those disclosed in U.S. Pat. No. 10,248,758 or U.S. Patent Pub. No. 2020/0392187 A1, the contents of which are incorporated by reference herein in their entireties.

The multimerization domains may be derived from a naturally-occurring protein sequence by substitution of at least one amino acid residue or by additional at the N- or C-terminus of one or more residues. In some cases, the first multimerization domain comprises a protein sequence determined by computational methods. This first multimerization domain may form the entire core of the VLP; or the core of the VLP may comprise one or more additional polypeptides (also referred to a “second component” or third, fourth, fifth component and so on), such that the VLP comprises two, three, four, five, six, seven, or more multimerization domains. In some cases, the first component will form trimers related by 3-fold rotational symmetry and the second component will form pentamers related by 5-fold rotational symmetry. In such cases, the VLP forms an “icosahedral particle” having I53 symmetry. Together these one or more pluralities of component may be arranged such that the members of each plurality of component are related to one another by symmetry operators. A general computational method for designing self-assembling protein materials, involving symmetrical docking of protein building blocks in a target symmetric architecture, is disclosed in U.S. Patent Pub. No. US 2015/0356240 A1.

The “core” of the VLP is used herein to describe the central portion of the VLP that links together the several copies of the RSV F protein ectodomain, or antigenic fragments thereof, displayed by the VLP. In an embodiment, the first component comprises a first polypeptide comprising an F protein, a linker, and a multimerization domain.

In some cases, the VLP is adapted to display the F protein from two or more diverse strains of RSV. In non-limiting examples, the same VLP displays mixed populations of protein antigens or mixed heterotrimers of protein antigens from different strains of RSV. The sequences of the F protein of various RSV strains are known in the art, see, e.g., NCBI Accession Nos.: QFX69124.1, QFX69112.1, APW78900.1, APW78889.1, APW78878.1, APW78867.1, APW78856.1, APW78845.1, APW78834.1, APW78823.1, APW78812.1, APW78801.1, APW78790.1, APW78779.1, APW78768.1, APW78757.1, APW78746.1, APW78735.1, APW78724.1, APW78713.1, APW78702.1, APW78691.1, APW78680.1, APW78669.1, APW78658.1, APW78647.1, APW78636.1, APW78625.1, APW78614.1, and AAR14266.1.

The VLPs of the present disclosure display antigenic proteins in various ways including as gene fusion or by other means disclosed herein. As used herein, “linked to” or “attached to” denotes any means known in the art for causing two polypeptides to associate. The association may be direct or indirect, reversible or irreversible, weak or strong, covalent or non-covalent, and selective or nonselective.

In some embodiments, attachment is achieved by genetic engineering to create an N- or C-terminus fusion of an antigen to one of the pluralities of polypeptides composing the VLP. Thus, the VLP may consist of, or consist essentially of, one, two, three, four, five, six, seven, eight, nine, or ten pluralities of polypeptides displaying one, two, three, four, five, six, seven, eight, nine, or ten pluralities of antigens, where at least one of the pluralities of antigen is genetically fused to at least one of the plurality of polypeptides. In some cases, the VLP consists essentially of one plurality of polypeptides capable of self-assembly and comprising the plurality of antigenic proteins genetically fused thereto. In some cases, the VLP consists essentially of a first plurality of polypeptides comprising a plurality of antigens; and a second plurality of polypeptides capable of co-assembling into two-component VLP, one plurality of polypeptides linking the antigenic protein to the VLP and the other plurality of polypeptides promoting self-assembly of the VLP.

In some embodiments, attachment is achieved by post-translational covalent attachment between one or more pluralities of polypeptides and one or more pluralities of antigenic protein. In some cases, chemical cross-linking is used to non-specifically attach the antigen to a VLP polypeptide. In some cases, chemical cross-linking is used to specifically attach the antigenic protein to a VLP polypeptide (e.g. to the first polypeptide or the second polypeptide). Various specific and non-specific cross-linking chemistries are known in the art, such as Click chemistry and other methods. In general, any cross-linking chemistry used to link two proteins may be adapted for use in the presently disclosed VLPs. In particular, chemistries used in creation of immunoconjugates or antibody drug conjugates may be used. In some cases, an VLP is created using a cleavable or non-cleavable linker. Processes and methods for conjugation of antigens to carriers are provided by, e.g., U.S. Patent Pub. No. US 2008/0145373 A1.

The components of the VLP of the present disclosure may have any of various amino acids sequences. U.S. Patent Pub No. US 2015/0356240 A1 describes various methods for designing protein assemblies. As described in US Patent Pub No. US 2016/0122392 A1 and in International Patent Pub. No. WO 2014/124301 A1, the polypeptides were designed for their ability to self-assemble in pairs to form VLPs, such as icosahedral particles. The design involved design of suitable interface residues for each member of the polypeptide pair that can be assembled to form the VLP. The VLPs so formed include symmetrically repeated, non-natural, non-covalent polypeptide-polypeptide interfaces that orient a first assembly and a second assembly into a VLP, such as one with an icosahedral symmetry.

Non-limiting examples of designed protein complexes useful in protein-based VLPs of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety. Illustrative sequences are provided in Table 1.

TABLE 1

| Component | | | |
|---|---|---|---|
| Type | Description | Mutations | Sequence |
| Comp A-RSV | DS-Cav1- | | QNITEEFYQSTCSAVSKGYLSALRTGWYTS |
| fusion | T33-15B | | VITIELSNIKENKCNGTDAKVKLIKQELDKY |
| | | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGGS |
| | | | MVRGIRGAITVNSDTPTSIIIATILLLEKMLEA |
| | | | NGIQSYEELAAVIFTVTEDLTSAFPAEAARQI |
| | | | GMHRVPLLSAREVPVPGSLPRVIRVLALWN |
| | | | TDTPQDRVRHVYLSEAVRLRPDLESAQ |
| | | | (SEQ ID NO: 1) |
| Comp A-RSV | DS-Cav1- | | QNITEEFYQSTCSAVSKGYLSALRTGWYTS |
| fusion | foldon-I53- | | VITIELSNIKENKCNGTDAKVKLIKQELDKY |
| | 50A | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRGYIPEAPRD |
| | | | GQAYVRKDGEWVLLSTFLGSGSHHHHHHH |
| | | | HGGSGGSGSEKAAKAEEAARKMEELFKKH |
| | | | KIVAVLRANSVEEAIEKAVAVFAGGVHLIEI |
| | | | TFTVPDADTVIKALSVLKEKGAIIGAGTVTS |
| | | | VEQCRKAVESGAEFIVSPHLDEEISQFCKEK |
| | | | GVFYMPGVMTPTELVKAMKLGHTILKLFPG |
| | | | EVVGPQFVKAMKGPFPNVKFVPTGGVNLD |
| | | | NVCEWFKAGVLAVGVGSALVKGTPDEVRE |
| | | | KAKAFVEKIRGCTE (SEQ ID NO: 2) |
| Comp A-RSV | DS-Cav1-I53- | | QNITEEFYQSTCSAVSKGYLSALRTGWYTS |
| fusion | 50A | | VITIELSNIKENKCNGTDAKVKLIKQELDKY |
| | | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRGGSGGSGSE |
| | | | KAAKAEEAARKMEELFKKHKIVAVLRANS |
| | | | VEEAIEKAVAVFAGGVHLIEITFTVPDADTV |
| | | | IKALSVLKEKGAIIGAGTVTSVEQCRKAVES |
| | | | GAEFIVSPHLDEEISQFCKEKGVFYMPGVMT |
| | | | PTELVKAMKLGHTILKLFPGEVVGPQFVKA |
| | | | MKGPFPNVKFVPTGGVNLDNVCEWFKAGV |
| | | | LAVGVGSALVKGTPDEVREKAKAFVEKIRG |
| | | | CTE (SEQ ID NO: 3) |
| Comp A-RSV | DS-Cav1-I32- | | QNITEEFYQSTCSAVSKGYLSALRTGWYTS |
| fusion | 28A | | VITIELSNIKENKCNGTDAKVKLIKQELDKY |
| | | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGGS |
| | | | GGSGSDDARIAAIGDVDELNSQIGVLLAEPL |
| | | | PDDVRAALSAIQHDLFDLGGELCIPGHAAIT |
| | | | EDHLLRLALWLVHYNGQLPPLEEFILPGGA |
| | | | RGAALAHVCRTVCRRAERSIKALGASEPLNI |
| | | | APAAYVNLLSDLLFVLARVLNRAAGGADV |
| | | | LWDRTRAH (SEQ ID NO: 4) |
| Comp A-RSV | DS-Cav1- | | QNITEEFYQSTCSAVSKGYLSALRTGWYTS |
| fusion | foldon-15GS- | | VITIELSNIKENKCNGTDAKVKLIKQELDKY |
| | HelExt-I53- | | KNAVTELQLLMQSTPATNNRARRELPRFMN |
| | 50A (F14) | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRGYIPEAPRD |
| | | | GQAYVRKDGEWVLLSTFLGSGGSGSGSGG |
| | | | SGSGEKAAKAEEAARKMEELFKKHKIVAVL |
| | | | RANSVEEAIEKAVAVFAGGVHLIEITFTVPD |
| | | | ADTVIKALSVLKEKGAIIGAGTVTSVEQCRK |
| | | | AVESGAEFIVSPHLDEEISQFCKEKGVFYMP |
| | | | GVMTPTELVKAMKLGHTILKLFPGEVVGPQ |
| | | | FVKAMKGPFPNVKFVPTGGVNLDNVCEWF |
| | | | KAGVLAVGVGSALVKGTPDEVREKAKAFV |
| | | | EKIRGCTE (SEQ ID NO: 5) |
| Comp A-RSV | CompA-RSV- | none | QNITEEFYQSTCSAVSKGYLSALRTGWYTS |
| fusion | 02 | | VITIELSNIKENKCNGTDAKVKLIKQELDKY |
| | | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGSG |
| | | | GSGSGSGGSEKAAKAEEAARKMEELFKKH |
| | | | KIVAVLRANSVEEAIEKAVAVFAGGVHLIEI |
| | | | TFTVPDADTVIKALSVLKEKGAIIGAGTVTS |
| | | | VEQCRKAVESGAEFIVSPHLDEEISQFCKEK |
| | | | GVFYMPGVMTPTELVKAMKLGHTILKLFPG |
| | | | EVVGPQFVKAMKGPFPNVKFVPTGGVNLD |
| | | | NVCEWFKAGVLAVGVGSALVKGTPDEVRE |
| | | | KAKAFVEKIRGCTE (SEQ ID NO: 6) |
| RSV F | DS-Cav1- | | QNITEEFYQSTCSAVSKGYLSALRTGWYTS |
| | foldon | | VITIELSNIKENKCNGTDAKVKLIKQELDKY |
| | | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGYI |
| | | | PEAPRDGQAYVRKDGEWVLLSTFL (SEQ ID |
| | | | NO: 7) |
| Comp A-RSV | DS-Cav1- | | QNITEEFYQSTCSAVSKGYLSALRTGWYTS |
| fusion | foldon-T33- | | VITIELSNIKENKCNGTDAKVKLIKQELDKY |
| | 31A | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGYI |
| | | | PEAPRDGQAYVRKDGEWVLLSTFLGGSME |
| | | | EVVLITVPSALVAVKIAHALVEERLAACVNI |
| | | | VPGLTSIYREEGSVVSDHELLLLVKTTTDAF |
| | | | PKLKERVKELHPYEVPEIVALPIAEGNREYL |
| | | | DWLRENTG (SEQ ID NO: 8) |
| Comp A-RSV | DS-Cav1- | | QNITEEFYQSTCSAVSKGYLSALRTGWYTS |
| fusion | T33-31A | | VITIELSNIKENKCNGTDAKVKLIKQELDKY |
| | | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGGS |
| | | | MEEVVLITVPSALVAVKIAHALVEERLAAC |
| | | | VNIVPGLTSIYREEGSVVSDHELLLLVKTTT |
| | | | DAFPKLKERVKELHPYEVPEIVALPIAEGNR |
| | | | EYLDWLRENTG (SEQ ID NO: 9) |
| Comp A-RSV | DS-Cav1- | | QNITEEFYQSTCSAVSKGYLSALRTGWYTS |
| fusion | foldon-T33- | | VITIELSNIKENKCNGTDAKVKLIKQELDKY |
| | 15B | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELLGYI |
| | | | PEAPRDGQAYVRKDGEWVLLSTFLGGSMV |
| | | | RGIRGAITVNSDTPTSIIIATILLLEKMLEANG |
| | | | IQSYEELAAVIFTVTEDLTSAFPAEAARQIG |
| | | | MHRVPLLSAREVPVPGSLPRVIRVLALWNT |
| | | | DTPQDRVRHVYLSEAVRLRPDLESAQ (SEQ |
| | | | ID NO: 10) |
| CompA | I53-50AΔcys | C74A, C98A, | MEELFKKHKIVAVLRANSVEEAIEKAVAVF |
| | | C163A, C201A | AGGVHLIEITFTVPDADTVIKALSVLKEKGA |
| | | | IIGAGTVTSVEQARKAVESGAEFIVSPHLDE |
| | | | EISQFAKEKGVFYMPGVMTPTELVKAMKL |
| | | | GHTILKLFPGEVVGPQFVKAMKGPFPNVKF |
| | | | VPTGGVNLDNVAEWFKAGVLAVGVGSALV |
| | | | KGTPDEVREKAKAFVEKIRGATE (SEQ ID |
| | | | NO: 30) |
| CompA | I53_dn5B | | EEAELAYLLGELAYKLGEYRIAIRAYRIALK |
| | | | RDPNNAEAWYNLGNAYYKQGRYREAIEYY |
| | | | QKALELDPNNAEAWYNLGNAYYERGEYEE |
| | | | AIEYYRKALRLDPNNADAMQNLLNAKMRE |
| | | | E (SEQ ID NO: 31) |
| Signal | | | MELLILKANAITTILTAVTFCFASG (SEQ ID |
| sequence | | | NO: 11) |
| Signal | | | MELLILKANVIATILTAVTFCFASS (SEQ ID |
| sequence | | | NO: 12) |
| Signal | | | MSWKVVIIFSLLITPQHG (SEQ ID NO: 13) |
| sequence | | | |
| RSV F | RSV F protein | S155C, S290C, | QNITEEFYQSTCSAVSKGYLSALRTGWYTS |
| | DS-Cav1 | S190F, V207L | VITIELSNIKENKCNGTDAKVKLIKQELDKY |
| | | | KNAVTELQLLMQSTPATNNRARRELPRFMN |
| | | | YTLNNAKKTNVTLSKKRKRRFLGFLLGVGS |
| | | | AIASGVAVCKVLHLEGEVNKIKSALLSTNK |
| | | | AVVSLSNGVSVLTFKVLDLKNYIDKQLLPIL |
| | | | NKQSCSISNIETVIEFQQKNNRLLEITREFSV |
| | | | NAGVTTPVSTYMLTNSELLSLINDMPITNDQ |
| | | | KKLMSNNVQIVRQQSYSIMCIIKEEVLAYVV |
| | | | QLPLYGVIDTPCWKLHTSPLCTTNTKEGSNI |
| | | | CLTRTDRGWYCDNAGSVSFFPQAETCKVQS |
| | | | NRVFCDTMNSLTLPSEVNLCNVDIFNPKYD |
| | | | CKIMTSKTDVSSSVITSLGAIVSCYGKTKCT |
| | | | ASNKNRGIIKTFSNGCDYVSNKGVDTVSVG |
| | | | NTLYYVNKQEGKSLYVKGEPIINFYDPLVFP |
| | | | SDEFDASISQVNEKINQSLAFIRKSDELL |
| | | | (SEQ ID NO: 14) |
| Antigen | RSV-B F | | QNITEEFYQSTCSAVSRGYFSALRTGWYTS |
| | protein | | VITIELSNITETKCNGTDTKVKLIKQELDKYK |
| | | | NAVTELQLLMQNTPAANNRARREAPQHMN |
| | | | YTINTTKNLNVSISKKRKRRFLGFLLGVGSA |
| | | | IASGIAVSKVLHLEGEVNKIKNALLSTNKAV |
| | | | VSLSNGVSVLTSKVLDLKNYINNQLLPIVNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMSIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELL (SEQ ID |
| | | | NO: 34) |
| Antigen | RSV-B F | | QNITEEFYQSTCSAVSRGYFSALRTGWYTS |
| | protein | | VITIELSNITETKCNGTDTKVKLIKQELDKYK |
| | (stabilized) | | NAVTELQLLMQNTPAANNRARREAPQHMN |
| | | | YTINTTKNLNVSISKKRKRRFLGFLLGVGSA |
| | | | IASGIAVCKVLHLEGEVNKIKNALLSTNKAV |
| | | | VSLSNGVSVLTFKVLDLKNYINNQLLPILNQ |
| | | | QSCRIFNIETVIEFQQKNSRLLEITREFSVNA |
| | | | GVTTPLSTYMLTNSELLSLINDMPITNDQKK |
| | | | LMSSNVQIVRQQSYSIMCIIKEEVLAYVVQL |
| | | | PIYGVIDTPCWKLHTSPLCTTNIKEGSNICLT |
| | | | RTDRGWYCDNAGSVSFFPQADTCKVQSNR |
| | | | VFCDTMNSLTLPSEVSLCNTDIFNSKYDCKI |
| | | | MTSKTDISSSVITSLGAIVSCYGKTKCTASN |
| | | | KNRGIIKTFSNGCDYVSNKGVDTVSVGNTL |
| | | | YYVNKLEGKNLYVKGEPIINYYDPLVFPSDE |
| | | | FDASISQVNEKINQSLAFIRKSDELL (SEQ ID |
| | | | NO: 35) |
| Linker | Gly-Ser | | GSGGSGSGSGGSGSG (SEQ ID NO: 15) |
| Linker | Gly-Ser | | GSGGSGSGSGGS (SEQ ID NO: 16) |
| Linker | Gly-Ser | | GGSGGSGS (SEQ ID NO: 17) |
| Linker | Gly-Ser | | GSGGSGSG (SEQ ID NO: 18) |
| Linker | Extension | | EKAAKAEEAAR (SEQ ID NO: 19). |
| Linker | Extension | | EKAAKAEEAARK (SEQ ID NO: 20) |
| Trimerization | Foldon | | GYIPEAPRDGQAYVRKDGEWVLLSTFL |
| | | | (SEQ ID NO: 21). |
| CompB | I53-50B | | MNQHSHKDYETVRIAVVRARWHAEIVDAC |
| | | | VSAFEAAMADIGGDRFAVDVFDVPGAYEIP |
| | | | LHARTLAETGRYGAVLGTAFVVNGGIYRHE |
| | | | FVASAVIDGMMNVQLSTGVPVLSAVLTPHR |
| | | | YRDSDAHTLLFLALFAVKGMEAARACVEIL |
| | | | AAREKIAA (SEQ ID NO: 22) |
| CompB | I53-50B.1 | | MNQHSHKDHETVRIAVVRARWHAEIVDAC |
| | | | VSAFEAAMRDIGGDRFAVDVFDVPGAYEIP |
| | | | LHARTLAETGRYGAVLGTAFVVNGGIYRHE |
| | | | FVASAVIDGMMNVQLDTGVPVLSAVLTPH |
| | | | RYRDSDAHTLLFLALFAVKGMEAARACVEI |
| | | | LAAREKIAA (SEQ ID NO: 23) |
| CompB | I53-50A | none | MEELFKKHKIVAVLRANSVEEAIEKAVAVF |
| | | | AGGVHLIEITFTVPDADTVIKALSVLKEKGA |
| | | | IIGAGTVTSVEQCRKAVESGAEFIVSPHLDEE |
| | | | ISQFCKEKGVFYMPGVMTPTELVKAMKLG |
| | | | HTILKLFPGEVVGPQFVKAMKGPFPNVKFV |
| | | | PTGGVNLDNVCEWFKAGVLAVGVGSALVK |
| | | | GTPDEVREKAKAFVEKIRGCTE (SEQ ID NO: |
| | | | 24) |
| CompB | I53- | | NQHSHKDHETVRIAVVRARWHAEIVDACV |
| | 50B.1NegT2 | | SAFEAAMRDIGGDRFAVDVFDVPGAYEIPL |
| | | | HARTLAETGRYGAVLGTAFVVDGGIYDHEF |
| | | | VASAVIDGMMNVQLDTGVPVLSAVLTPHE |
| | | | YEDSDADTLLFLALFAVKGMEAARACVEIL |
| | | | AAREKIAA (SEQ ID NO: 25) |
| CompB | I53- | H9Y, R38A, | MNQHSHKDHETVRIAVVRARWHAEIVDAC |
| | 50B.4PosT1 | N96D, N105S, | VSAFEAAMRDIGGDRFAVDVFDVPGAYEIP |
| | | N109R, D111R, | LHARTLAETGRYGAVLGTAFVVNGGIYRHE |
| | | K122D, K124D | FVASAVINGMMNVQLNTGVPVLSAVLTPH |
| | | | NYDKSKAHTLLFLALFAVKGMEAARACVEI |
| | | | LAAREKIAA (SEQ ID NO: 26) |
| CompB | T33-15B | | MVRGIRGAITVNSDTPTSIIIATILLLEKMLEA |
| | | | NGIQSYEELAAVIFTVTEDLTSAFPAEAARQI |
| | | | GMHRVPLLSAREVPVPGSLPRVIRVLALWN |
| | | | TDTPQDRVRHVYLSEAVRLRPDLESAQ |
| | | | (SEQ ID NO: 27) |
| CompB | I32-28A | | MGDDARIAAIGDVDELNSQIGVLLAEPLPD |
| | | | DVRAALSAIQHDLFDLGGELCIPGHAAITED |
| | | | HLLRLALWLVHYNGQLPPLEEFILPGGARG |
| | | | AALAHVCRTVCRRAERSIKALGASEPLNIAP |
| | | | AAYVNLLSDLLFVLARVLNRAAGGADVLW |
| | | | DRTRAH (SEQ ID NO: 28) |
| CompB | T33-31A | | MEEVVLITVPSALVAVKIAHALVEERLAAC |
| | | | VNIVPGLTSIYREEGSVVSDHELLLLVKTTT |
| | | | DAFPKLKERVKELHPYEVPEIVALPIAEGNR |
| | | | EYLDWLRENTG (SEQ ID NO: 29) |
| CompB | I53_dn5A | | KYDGSKLRIGILHARWNAEIILALVLGALKR |
| | | | LQEFGVKRENIIIETVPGSFELPYGSKLFVEK |
| | | | QKRLGKPLDAIIPIGVLIKGSTMHFEYICDST |
| | | | THQLMKLNFELGIPVIFGVLTCLTDEQAEAR |
| | | | AGLIEGKMHNHGEDWGAAAVEMATKFN |
| | | | (SEQ ID NO: 32) |

In some embodiments, the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 24 and 30-31.

In some embodiments, the second multimerization domain comprises an amino acid sequence which is at least 75%, 80%, 85%, 90%, 91%, 92%, 93%, 94%, 95%, 96%, 97%, 98%, 99%, or 100% identical to an amino acid sequence of SEQ ID NO: 22-23 and 25-26.

The 50A first multimerization domains pair with the 50B second multimerization domains. The dn5B first multimerization domains pair with the dn5A second multimerization domains.

In some embodiments, the VLP comprises a fusion protein that has at least 95%, at least 96%, at least 97%, at least 98%, at least 99%, or 100% identity to any one of SEQ ID NO: 1-10 and comprises an RSV F protein as disclosed herein; and a second component that has at least 95%, at least 96%, at least 97%, at least 98%, at least 99%, or 100% identity to any one of SEQ ID NO: 22-23, 25-29, and 32.

In some embodiments, the VLP comprises a fusion protein that has at least 95%, at least 96%, at least 97%, at least 98%, at least 99%, or 100% identity to SEQ ID NO: 6 and comprises an RSV F protein as disclosed herein; and a second component that has at least 95%, at least 96%, at least 97%, at least 98%, at least 99%, or 100% identity to SEQ ID NO: 26.

In some embodiments, the first component comprises the polypeptide sequence that has at least 95%, at least 96%, at least 97%, at least 98%, at least 99%, or 100% identity to any one of SEQ ID NOs: 14, 34, and 35.

The first component may comprise an RSV F protein, which may be a full-length RSV F protein, the ectodomain of RSV F, or an antigenic fragment thereof. In some embodiments, the RSV F protein comprises the polypeptide sequence that has at least 95%, at least 96%, at least 97%, at least 98%, at least 99%, or 100% identity to any one of SEQ ID NOs: 14, 34, and 35. In some embodiments, the RSV F protein is an RVS-A F protein. In some embodiments, the RSV F protein is an RSV-B F protein.

In some embodiments, the first component comprises the polypeptide sequence that has at least 95%, at least 96%, at least 97%, at least 98%, at least 99%, or 100% identity to SEQ ID NOs: 14 and further comprises a signal peptide. In some embodiments, the signal peptide comprises the sequence of any one of SEQ ID NOs: 11-13.

The polypeptides as described herein may have one of more amino acid substitutions relative to wildtype RSV. For example and without limitation, the second component of a protein complex provided herein may comprise 1, 2, 3, 4, 5, 6, 7, or all 8 positions relative to SEQ ID NO: 14 selected from any one of H9Y, E24D, A28Q, A36E, R38A, N97D, N105S, D121H,

A polypeptide provided herein may comprise one or more conservative amino acid substitutions. The terminology “conservative amino acid substitution” is well known in the art and relates to substitution of a particular amino acid by one having a similar characteristic (e.g., similar charge or hydrophobicity). Conservative mutations can include, without limitation, substitution of amino acid residues with e.g., similar charge or hydrophobicity but differing in size or bulkiness (e.g., to provide a cavity-filling function). A list of conservative amino acid substitutions is given in the table below.

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, Met, Ile, D-Met, D- |
| | | Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, B-Ala, Acp |
| Isoleucine | I | D-Ile, Val, D-Val, AdaA, AdaG, Leu, D-Leu, Met, D-Met |
| Leucine | L | D-Leu, Val, D-Val, AdaA, AdaG, Leu, D-Leu, Met, D-Met |
| Lysine | K | D-Lys, Arg, D-Arg, homo-Arg, D-homo-Arg, Met, D-Met, Ile, D- |
| | | Ile, Orn, D-Orn |
| Methionine | M | D-Met, S—Me-Cys, Ile, D-Ile, Leu, D-Leu, Val, D-Val |
| Phenylalanine | F | D-Phe, Tyr, D-Thr, L-Dopa, His, D-His, Trp, D-Trp, Trans-3,4 or 5- |
| | | phenylproline, AdaA, AdaG, cis-3,4 or 5-phenylproline, Bpa, D- |
| | | Bpa |
| Proline | P | D-Pro, L-I-thioazolidine-4-carboxylic acid, D-or-L-1-oxazolidine-4- |
| | | carboxylic acid (Kauer, U.S. Pat. No. (4,511,390) |
| Serine | S | D-Ser, Thr, D-Thr, allo-Thr, Met, D-Met, Met (O), D-Met (O), L- |
| | | Cys, D-Cys |
| Threonine | T | D-Thr, Ser, D-Ser, allo-Thr, Met, D-Met, Met (O), D-Met (O), Val, |
| | | D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, AdaG |

Alternatively, a non-conservative amino acid substitution may be preferred, for example, for eradication of a flexible portion of the native RSV F protein secondary structure is desired, for example, by adding a cysteine residue (or vice versa). “Non-conservative substitution” refers to the substitution of an amino acid in one class with an amino acid from another class; for example, substitution of an Ala with Asp, Asn, Glu, or Gln. Additional non-limiting examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue. Substitutions of D-Cys for D-Ala, D-Ser, or D-Tyr (or another residue) may be used to remove intramolecular disulfide bonds, which may, in some cases improve protein stability or expression. Substitutions to D-Cys may be used to generated disulfide bonds that stability a protein or lock a protein into a desired conformation.

### Nucleic Acids, Vectors, and Cells

In another aspect, the disclosure provides nucleic acids encoding a polypeptide or fusion protein of the disclosure. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In another aspect, disclosure provides expression vectors comprising the isolated nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In another aspect, the present disclosure provides cells comprising the polypeptide, the virus-like particle, the composition, the nucleic acid, and/or the expression vector of any embodiment or combination of embodiments of the disclosure, wherein the cells can be either prokaryotic or eukaryotic, such as mammalian cells. In some embodiments the cells may be transiently or stably transfected with the nucleic acids or expression vectors of the disclosure. Such transfection of expression vectors into prokaryotic and eukaryotic cells can be accomplished via any technique known in the art. A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

### Pharmaceutical Compositions

In another aspect, the disclosure provides pharmaceutical compositions/vaccines comprising
(a) the polypeptide, the virus-like particle, the composition, the nucleic acid, the expression vector, and/or the cell of embodiment or combination of embodiments herein; and
(b) a pharmaceutically acceptable carrier.

As shown in the examples that follow, the virus-like particles elicit potent and protective antibody responses against RSV (e.g., against RSV-A and/or RSV-B). For example, the virus-like particles of the disclosure induce neutralizing antibody titers.

In some embodiments, a pharmaceutical composition or vaccine provided herein may be bivalent, e.g., may comprise both RSV-A and RSV-B antigens (e.g., both RSV-A and RSV-B F proteins).

The compositions/vaccines may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Illustrative tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Illustrative stabilizers include sucrose, sorbitol, glycine, inositol, sodium chloride, methionine, arginine, and arginine hydrochloride.

The virus-like particles may be the sole active agent in the composition, e.g., formulated as an aqueous vaccine, or the composition may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen.

Illustrative types of adjuvants that may be used in a pharmaceutical composition provided herein include the following: 1. mineral-containing compositions; 2. oil emulsions; 3. saponin formulations; 4. virosomes and virus-like particles; 5. bacterial or microbial derivatives; 6. bioadhesives and mucoadhesives; 7. liposomes; 8. polyoxyethylene ether and polyoxyethylene ester formulations; 9. polyphosphazene (pcpp); 10. muramyl peptides; 11. imidazoquinolone compounds; 12. thiosemicarbazone compounds; 13. tryptanthrin compounds; 14. human immunomodulators; 15. lipopeptides; 16. benzonaphthyridines; 17. microparticles; 18. immunostimulatory polynucleotide (such as RNA or DNA; e.g., cpg-containing oligonucleotides).

Illustrative adjuvants that may be used in a pharmaceutical composition provided herein include, but are not limited to, 3M-052, Adju-Phos™, Alhydrogel™, Adjumer™, albumin-heparin microparticles, Algal Glucan, Algammulin, Alum, Antigen Formulation, AS-2 adjuvant, ASO1, ASO3, autologous dendritic cells, autologous PBMC, Avridine™, B7-2, BAK, BAY R1005, BECC TLR-4 agonists, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CPG-1018, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, Matrix-M™ adjuvant, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribi like adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, SWE, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used. In some embodiments, the adjuvant is an aluminum hydroxide gel (e.g., Alhydrogel™). In some embodiments, the adjuvant is SWE. In some embodiments, the adjuvant is MF59. In some embodiments, the adjuvant is an oil-in-water emulsion.

For example, the composition may include an aluminum salt adjuvant, an oil in water emulsion (e.g. an oil-in-water emulsion comprising squalene, such as MF59, SWE, or AS03), a TLR9 agonist (such as CpG oligodeoxynucleotides), a TLR7 agonist (such as imidazoquinoline or imiquimod), or a combination thereof. In some embodiments, the adjuvant is a combination of an aluminum salt and CPG1018. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g. gel, crystalline, amorphous, etc.), with adsorption of antigen to the salt being an example. The concentration of Al⁺⁺⁺ in a composition for administration to a patient may be less than 5 mg/ml e.g. <4 mg/ml, <3 mg/ml, <2 mg/ml, <1 mg/ml, etc. An illustrative range is between 0.3 and 1 mg/ml. Aluminum hydroxide and aluminum phosphate adjuvants are suitable for use with the disclosure. In some embodiments, a pharmaceutical composition provided herein comprises aluminum hydroxide as an adjuvant. In some embodiment, a pharmaceutical composition provided herein comprises 500 μg aluminum hydroxide.

In some embodiments, the composition including the virus-like particles may be the sole active agent in the composition, where no adjuvant is included, or wherein the composition is substantially free of an adjuvant, including substantially free of any adjuvant. For example, no adjuvant may be added, or substance(s) having adjuvant property present but minimal quantities, such as quantities not expected to exert an adjuvant effect. In some embodiments, the pharmaceutical composition has less than about 5%, less than about 4%, less than about 4%, less than about 3%, less than about 2%, less than about 1%, less than about 0.5%, less than about 0.1%, less than 5%, less than 4%, less than 4%, less than 3%, less than 2%, less than 1%, less than 0.5%, or less than 0.1% (w/v) of the adjuvant. In some embodiments, the composition including the virus-like particles may be the sole active agent in the composition and is free of an adjuvant, (e.g, Alum or aluminum salt adjuvants).

Also provided herein are unit doses of the pharmaceutical composition described herein. In some embodiments, the unit dose comprises about 1 μg to about 5 μg, about 5 μg to about 10 μg, about 10 μg to about 15 μg, about 15 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 200 μg, about 200 μg to about 250 μg, about 250 μg to about 300 μg, about 300 μg to about 350 μg, about 350 μg to about 400 μg, about 400 μg to about 450 μg, or about 450 μg to about 500 μg of protein complex. In some embodiments, the unit dose comprises about 1 μg, about 2 μg, about 5 μg, about 10 μg, about 15 μg, about 25 μg, about 50 μg, about 75 μg, about 100 μg, about 125 μg, about 150 μg, about 200 μg, or about 250 μg of the protein complex. In some embodiments, the unit dose comprises, 25 μg, 75 μg, or 250 μg of the protein complex. The abbreviation μg may be used interchangeably with the abbreviation mcg to refer to micrograms of a substance. In some embodiments, the unit dosage comprises 5 μg of the protein complex. In some embodiments, the unit dosage comprises 25 μg of the protein complex. In some embodiments, the unit dosage comprises 125 μg of the protein complex. In some embodiments, the unit dosage comprises 100 μg of the protein complex. In another aspect, provided herein is a unit dose of the pharmaceutical composition described herein, wherein the unit dose comprises 2 μg, 5 μg, 10 μg, 15 μg, 25 μg, 50 μg, 75 μg, 100 μg, or 125 μg of the protein complex. In some embodiments, provided herein is a unit dose of the pharmaceutical composition described herein, wherein the unit dose comprises between about 25 μg and about 125 μg of the protein complex. In some embodiments, the unit dose of the pharmaceutical composition is between about 2 μg to about 125 μg, or between about 5 μg to about 125 g, or between about 15 μg to 125 μg, or between about 25 μg to about 125 μg, or between about 50 μg to about 125 μg, or between about 100 μg to about 125 μg of the protein complex.

In some embodiments, provided herein is a unit dose of the pharmaceutical composition described herein, wherein the unit dose comprises between about 25 μg and about 125 μg of the protein complex. In some embodiments, the unit dose of the pharmaceutical composition is between about 2 μg to about 125 μg, or between about 5 μg to about 125 g, or between about 15 μg to 125 μg, or between about 25 μg to about 125 μg, or between about 50 μg to about 125 μg, or between about 100 μg to about 125 μg of the protein complex.

In some embodiments, about 10 μg to about 100 μg, about 10 μg to about 150 μg, about 10 μg to about 200 μg, about 10 μg to about 250 μg, about 10 μg to about 300 μg, about 10 μg to about 350 μg, about 10 μg to about 400 μg, about 10 μg to about 450 μg, or about 10 μg to about 500 μg of the protein complex are administered.

In some embodiments, about 25 μg to about 100 μg, about 25 μg to about 150 μg, about 25 μg to about 200 μg, about 25 μg to about 250 μg, about 25 μg to about 300 μg, about 25 μg to about 350 μg, about 25 μg to about 400 μg, about 25 μg to about 450 μg, or about 25 μg to about 500 μg of the protein complex are administered.

In some embodiments, about 50 μg to about 100 μg, about 50 μg to about 150 μg, about 50 μg to about 200 μg, about 50 μg to about 250 μg, about 50 μg to about 300 μg, about 50 μg to about 350 μg, about 50 μg to about 400 μg, about 50 μg to about 450 μg, or about 50 μg to about 500 μg of the protein complex are administered.

In some embodiments, about 5 μg to about 150 μg, about 10 μg to about 150 μg, about 25 μg to about 150 μg, about 50 μg to about 150 μg, about 75 μg to about 150 μg, about 100 μg to about 150 μg, or about 125 μg to about 150 μg of the protein complex are administered.

In some embodiments, about 5 μg to about 125 μg, about 10 μg to about 125 μg, about 25 μg to about 125 μg, about 50 μg to about 125 μg, about 75 μg to about 125 μg, or about 100 μg to about 125 μg of the protein complex are administered.

In some embodiments, about 5 μg to about 100 μg, about 10 μg to about 100 μg, about 25 μg to about 100 μg, about 50 μg to about 100 μg, or about 75 μg to about 100 μg of the protein complex are administered.

In some embodiments, about 5 μg to about 75 μg, about 10 μg to about 75 μg, about 25 μg to about 75 μg, or about 50 μg to about 75 μg of the protein complex are administered.

In some embodiments, about 5 μg to about 50 μg, about 10 μg to about 50 μg, or about 25 μg to about 50 μg of the protein complex are administered.

The protein complexes of the disclosure comprise an RSV F protein. In illustrative embodiments, the total molecular mass of the protein complex is about 5.6 MDa and the mass fraction of the RSV F protein in the protein complex is about 58%; there are 20 copies of the RSV F protein trimer per protein complex in these embodiments. Accordingly, for these illustrative embodiments, the dose amounts described above may be converted to a molar amount, where 1 μg equals about 0.18 picomoles (pmol) of the protein complex or about 3.6 pmol of RSV F protein within the protein complex. Each 1 μg of protein complex equals about 0.6 μg of RSV F protein. A unit dose of 25 μg of protein complex is equal to a unit dose of about 14 μg of a non-particle-associated RSV F protein trimer; conversely, a unit dose of about 100 μg of a non-particle-associated RSF F protein trimer is equivalent to about 174 μg of such an illustrative protein complex.

The pH of the formulation can also vary. In general, it is between about pH 6.2 to about pH 8.0. In some embodiments, the pH is about 6.2, about 6.4, about 6.6, about 6.8, about 7.0, about 7.2, about 7.4, about 7.6, about 7.8, or about 8.0. Of course, the pH may also be within a range of values. Thus, in some embodiments the pH is between about 6.2 and about 8.0, between about 6.2 and 7.8, between about 6.2 and 7.6, between about 6.2 and 7.4, between about 6.2 and 7.2, between about 6.2 and 7.0, between about 6.2 and 6.8, between about 6.2 and about 6.6, or between about 6.2 and 6.4. In other embodiments, the pH is between 6.4 and about 8.0, between about 6.4 and 7.8, between about 6.4 and 7.6, between about 6.4 and 7.4, between about 6.4 and 7.2, between about 6.4 and 7.0, between about 6.4 and 6.8, or between about 6.4 and about 6.6. In still other embodiments, the pH is between about 6.6 and about 8.0, between about 6.6 and 7.8, between about 6.6 and 7.6, between about 6.6 and 7.4, between about 6.6 and 7.2, between about 6.6 and 7.0, or between about 6.6 and 6.8. In yet other embodiments, it is between about 6.8 and about 8.0, between about 6.8 and 7.8, between about 6.8 and 7.6, between about 6.8 and 7.4, between about 6.8 and 7.2, or between about 6.8 and 7.0. In still other embodiments, it is between about 7.0 and about 8.0, between about 7.0 and 7.8, between about 7.0 and 7.6, between about 7.0 and 7.4, between about 7.0 and 7.2, between about 7.2 and 8.0, between about 7.2 and 7.8, between about 7.2 and about 7.6, between about 7.2 and 7.4, between about 7.4 and about 8.0, about 7.4 and about 7.6, or between about 7.6 and about 8.0.

In some embodiments, the formulation can include one or more salts, such as sodium chloride, sodium phosphate, or a combination thereof. In general, each salt is present in the formulation at about 10 mM to about 200 mM. Thus, in some embodiments, any salt that is present is present at about 10 mM to about 200 mM, about 20 mM to about 200 mM, about 25 mM to about 200 mM, at about 30 mM to about 200 mM, at about 40 mM to about 200 mM, at about 50 mM to about 200 mM, at about 75 mM to about 200 mM, at about 100 mM to about 200 mM, at about 125 mM to about 200 mM, at about 150 mM to about 200 mM, or at about 175 mM to about 200 mM. In other embodiments, any salt that is present is present at about 10 mM to about 175 mM, about 20 mM to about 175 mM, about 25 mM to about 175 mM, at about 30 mM to about 175 mM, at about 40 mM to about 175 mM, at about 50 mM to about 175 mM, at about 75 mM to about 175 mM, at about 100 mM to about 175 mM, at about 125 mM to about 175 mM, or at about 150 mM to about 175 mM. In still other embodiments, any salt that is present is present at about 10 mM to about 150 mM, about 20 mM to about 150 mM, about 25 mM to about 150 mM, at about 30 mM to about 150 mM, at about 40 mM to about 150 mM, at about 50 mM to about 150 mM, at about 75 mM to about 150 mM, at about 100 mM to about 150 mM, or at about 125 mM to about 150 mM. In yet other embodiments, any salt that is present is present at about 10 mM to about 125 mM, about 20 mM to about 125 mM, about 25 mM to about 125 mM, at about 30 mM to about 125 mM, at about 40 mM to about 125 mM, at about 50 mM to about 125 mM, at about 75 mM to about 125 mM, or at about 100 mM to about 125 mM. In some embodiments, any salt that is present is present at about 10 mM to about 100 mM, about 20 mM to about 100 mM, about 25 mM to about 100 mM, at about 30 mM to about 100 mM, at about 40 mM to about 100 mM, at about 50 mM to about 100 mM, or at about 75 mM to about 100 mM. In yet other embodiments, any salt that is present is present at about 10 mM to about 75 mM, about 20 mM to about 75 mM, about 25 mM to about 75 mM, at about 30 mM to about 75 mM, at about 40 mM to about 75 mM, or at about 50 mM to about 75 mM. In still other embodiments, any salt that is present is present at about 10 mM to about 50 mM, about 20 mM to about 50 mM, about 25 mM to about 50 mM, at about 30 mM to about 50 mM, or at about 40 mM to about 50 mM. In other embodiments, any salt that is present is present at about 10 mM to about 40 mM, about 20 mM to about 40 mM, about 25 mM to about 40 mM, at about 30 mM to about 40 mM, at about 10 mM to about 30 mM, at about 20 mM to about 30, at about 25 mM to about 30 mM, at about 10 mM to about 25 mM, at about 20 mM to about 25 mM, or at about 10 mM to about 20 mM. In some embodiments, the sodium chloride is present in the formulation at about 100 mM. In some embodiments, the sodium phosphate is present in the formulation at about 25 mM.

Formulations herein may further comprise a solubilizing agent such as a nonionic detergent. Such detergents include, but are not limited to polysorbate 80 (Tween® 80), TritonX100 and polysorbate 20.

In some embodiments, a pharmaceutical composition described herein may comprise a polypeptide, a virus-like particle, composition, a nucleic acid, an expression vector, and/or a cell of embodiment or combination of embodiments herein and one or more additional vaccines, as well as a pharmaceutically acceptable carrier. In some embodiments, the one or more additional vaccines is a pediatric vaccine. Vaccines that may be co-formulated with a the polypeptide, the virus-like particle, the composition, the nucleic acid, the expression vector, and/or the cell of embodiment or combination described herein include, without limitation, a vaccine against Hepatitis B, a vaccine against Rotavirus, a vaccines against diphtheria, tetanus and pertussis (“DTaP”), a vaccine against polio, a vaccine against influenza, and a vaccine against measles, mumps and rubella (“MMR”).

### Methods of Treatment

In another aspect, the disclosure provides methods to vaccinate a subject against infection with RSV (e.g., infection with RSV-A and/or RSV-B), comprising administering to a subject in need thereof an amount effective to treat or limit development of the infection of the polypeptide, virus-like particle, composition, nucleic acid, pharmaceutical composition, or vaccine of any embodiment herein (referred to as the “immunogenic composition”). In some embodiments, such a method prevents disease following infection with RSV subtypes A and/or B. In some embodiments, such a method protects against the development of RSV-associated disease (e.g., severe disease), for example, pneumonia and/or acute respiratory disease. The subject may be any suitable mammalian subject, including but not limited to a human subject. In some embodiments, a subject is a human child, e.g., a child of less than 12 months of age. In some embodiments, a subject is a human toddler, e.g., of about 1 to about 3 years of age or of about 1 to about 5 years of age. In some embodiment, the subject is a human adult of more than 60 years of age. In some embodiments, the subject is a human adult of more than 65 years of age. In particular embodiments, the subject is dependent on the help of others or with serious health concerns or risks (e.g. a frail elderly person). In some embodiments, the subject is a healthy adult of 18-60 years of age. In some embodiments, the subject is a healthy adult of 18-45 years of age. In another embodiment, the subject is a pregnant woman. In some embodiments the subject is an immunocompromised human adult. In some embodiments, the subject is a human adult suffering from chronic underlying heart and/or lung disease or from functional disability. In some embodiments, the subject is at risk of severe RSV disease (e.g., LRTI or pneumonia).

The immunogenic compositions provided herein may be used vaccinate an unborn child. The administration of certain inactivated vaccines are recommended during pregnancy to induce immunity in the unborn child, for example, the tetanus toxoid, reduced diphtheria toxoid and acellular pertussis (Tdap) vaccine and the influenza vaccine. Thus, in some embodiments, provide herein is a method of generating an immune response in an unborn child, the method comprising administering an effective amount of the immunogenic composition provided herein to the mother of said unborn child. The immunogenic composition may be administered at any suitable time point in the pregnancy, e.g., in the last trimester of the pregnancy.

An immunogenic composition provided herein may be co-administered with other treatments, such as other vaccines. Thus in some embodiments, a subject treated in accordance with a method provided herein may also be administered one or more seasonal or pandemic vaccines such as an influenza vaccine or a SARS-Cov2 vaccine. In some embodiments, a subject treated in accordance with the methods provided herein may also be administered a pneumococcal, Recombinant Zoster (Shingles), or Tdap vaccine. One, two, or more vaccines may be co-administered with an immunogenic composition provided herein. “Co-administration” includes both concurrently as well as subsequent administration. For example, the one, two, or more vaccines and an immunogenic composition provided herein may be administered on the same day. In some embodiments, the one, two, or more vaccines and an immunogenic composition provided herein are administered within 1 hour, within 2 hours, within 3 hours, within 4 hours, within 5 hours, within 6 hours, within 8 hours, within 10 hours, or within 12 hours.

In another aspect, provide herein is a method of treating a subject suffering from RSV infection. As used herein, “treat” or “treating” includes, but is not limited to accomplishing one or more of the following: (a) reducing RSV titer (e.g., RSV-A titer and/or RSV-B titer) in the subject; (b) limiting any increase of RSV titer (e.g., RSV-A titer and/or RSV-B titer) in the subject; (c) reducing the severity of RSV infection symptoms (e.g., RSV-A infection and/or RSV-B infection); (d) limiting or preventing development of symptoms after RSV infection (e.g., RSV-A infection and/or RSV-B infection); (e) inhibiting worsening of symptoms of RSV infection (e.g., RSV-A infection and/or RSV-B infection); (f) limiting or preventing recurrence of symptoms of RSV infection (e.g., RSV-A infection and/or RSV-B infection) in subjects that were previously symptomatic for RSV infection (e.g., RSV-A infection and/or RSV-B infection); and/or (e) survival. In some embodiments, a method of vaccinating decreases the subject's risk of becoming infected with RSV (e.g., RSV-A and/or RSV-B). In some embodiments, a method of vaccinating limits the development of an RSV infection (e.g., RSV-A infection and/or RSV-B infection). In some embodiments, a method of vaccinating decreases the severity of the symptoms of an RSV infection (e.g., RSV-A infection and/or RSV-B infection). In some embodiments, the infection with RSV (e.g., infection with RSV-A and/or RSV-B) is a lower respiratory tract infection (LRTI).

In some embodiments, the methods provided herein may be used to prevent an RSV infection or illness (e.g., pneumonia or acute respiratory disease) in a subject. As used herein, “prevent” or “preventing” includes, but is not limited to accomplishing one or more of the following: (a) generating an immune response (antibody and/or cell-based, e.g., CD4 T cells, memory B cells, and/or CD8 T cells) to RSV (e.g., RSV-A and/or RSV-B) in the subject expected to confer protection against lower respiratory tract infection (LRTI) caused by or associated with RSV in the subject; (b) generating neutralizing antibodies against RSV (e.g., RSV-A and/or RSV-B) in the subject expected to reduce the severity of LRTI caused by or associated with RSV in the subject; (c) preventing LRTI caused by or associated with RSV in a subject, detected as an increase in the titer of the virus i of the subject or by an increase in one or more symptoms of RSV infection; (d) preventing severe LRTI caused by or associated with RSV in a subject detected as an increase in the titer of the virus of the subject or by an increase in one or more severe symptoms of RSV infection; (e) reducing the risk of LRTI or severe LRTI caused by or associated with RSV within a population of subjects; or (f) causing a seroresponse (or seroconversion) of a subject, such as generating neutralizing antibodies against RSV at least 4-fold higher than a baseline antibody level in the subject. Prevention may be assessed by comparing immune responses, especially correlates of protection, in subjects administered a vaccine to the same subjects before administration (termed baseline), subjects administered a placebo, or subjects administered a comparator vaccine.

As used herein, “limiting” the development an RSV infection (e.g., RSV-A infection and/or RSV-B infection), refers to accomplishing one or more of the following: (a) generating an immune response (antibody and/or cell-based, e.g., CD4 T cells, memory B cells, and/or CD8 T cells) to RSV (e.g., RSV-A and/or RSV-B) in the subject expected to limit an increase in viral titer or symptoms in the subject; (b) generating neutralizing antibodies against RSV (e.g., RSV-A and/or RSV-B) in the subject at a level expected to limit an increase in viral titer or symptoms in the subject; (c) causing reduced RSV titers (e.g., RSV-A and/or RSV-B titers) in the subject after exposure to RSV (e.g., RSV-A and/or RSV-B) compared to subjects not administered the protein complex; and (d) caused reduced incidence or severity of symptoms after RSV infection (e.g., RSV-A infection and/or RSV-B infection). Illustrative symptoms of RSV infection include, but are not limited to, fever, fatigue, cough, nasal congestion, sneezing, shortness of breath, wheezing, and lower respiratory tract infections.

The methods provided herein may be used to prevent or limit development of infection with an RSV-A subtype and/or an RSV-B subtype.

Further, the methods provided herein may be used to prevent or limit development of infection with an original strain of RSV and/or infection with a variant strain of RSV. Examples of variant RSV strains include, without limitation, RSV ON1, RSV NA1, RSV LBA1, RSV LBA2, RSV BA, RSV Long, RAV A2, and others (see, e.g., Pandya et al., Pathogens 2019, 8(2), 67; and Melero and Moore, Curr Top Microbiol Immunol. 2013; 372: 59-82). The pharmaceutical composition of the present invention may be effective in preventing or limiting infection of RSV strains that have not yet been described or discovered.

Clinical efficacy of a respiratory virus vaccine can be assessed by various means known in the art, including but not limited to placebo-controlled clinical efficacy studies to measure viral load or symptoms of RSV disease in vaccinated versus control subjects. Correlates of protection may also be defined, such as neutralizing antibody titers (typically expressed as geometric mean titers), fold increases above baselines (typically expressed as geometric fold rise), and seroresponse rate (a percentage of subjects that achieve a fold rise in neutralizing antibody titers above a predetermined threshold). Guidance on direct and surrogate measures of clinical efficacy for respiratory diseases are available, for example, in Guidance for Industry: Clinical Data Needed to Support the Licensure of Seasonal Inactivated Influenza Vaccines. U.S. Food & Drug Administration (May 2007) and Respiratory Syncytial Virus Infection: Developing Antiviral Drugs for Prophylaxis and Treatment Guidance for Industry. U.S. Food & Drug Administration (October 2017).

In some embodiments, the methods described herein generate an immune response in a subject in the subject not known to be infected with RSV (e.g., RSV-A and/or RSV-B), wherein the immune response serves to limit development of infection and symptoms of an RSV (e.g., RSV-A and/or RSV-B) infection. In some embodiments, the immune response comprises generation of neutralizing antibodies and/or cell-based responses against RSV (e.g., RSV-A and/or RSV-B). In some embodiments, the immune response comprises generation of RSV F protein-specific (e.g., RSV-A and/or RSV-B F protein-specific) responses with a mean geometric titer of at least 1×10³, at least 1×10⁴, at least 1×10⁵, at least 1×10⁶, at least 1×10⁷, at least 1×10⁸, or at least 1×10⁹. In a further embodiment, the immune response comprises generation of antibodies against multiple antigenic epitopes or RSV (e.g., RSV-A and/or RSV-B).

In one aspect, the methods provided herein may results in an increase in antibody titers in a subject, e.g., in an increase in RSV-A-specific neutralizing antibodies, RSV-B-specific neutralizing antibodies, RSV F-protein-specific IgG antibodies, and/or RSV F-protein-specific neutralizing antibodies. Antibody titers may be determined using any suitable assays known in the art or described herein including, without limitation, binding enzyme-linked immunosorbent assays (ELISA), enzyme-linked immune absorbent spot (ELISpot), competition ELISAs, immunoprecipitation, immunoblotting, and agglutination assays.

In some embodiments, a neutralization or microneutralization (MN) assay may be used to measure increases in neutralizing antibodies in a subject after administration of a protein complex as described herein. Microneutralization refers to a neutralizing performed in a miniaturized format, such as a 96-well plate. A (micro)neutralization assay is used to test for the inhibition of a virus by antibodies (e.g., purified antibodies, serum, or plasma). The assay measures the level of antibodies present in a sample that are able to neutralize a virus in vitro. Generally, microneutralization assays for clinical samples are performed with a serial dilution of serum mixed with a fixed concentration of virus. Methods for performing (micro)neutralization assays are well known. Illustrative microneutralization assays are described, see, e.g., van Baalen et al. Vaccine 35 (2017) 46-52.

If neutralizing antibodies specific for RSV are present in a sample, the virus will be neutralized, and infection of cells (e.g., HEp-2 cells) is inhibited. Immunofluorescence levels indicating viral infection may analyzed using, e.g., a CTL ImmunoSpot® UV analyzer, equipped with BioSpot® analysis software for automated counting of infected cells. Results are generally reported in international units per milliliter (IU/mL). Validation and standardized of microneutralization assays is described in the Examples below.

In some embodiments, the methods provided herein result in an increase in antibodies (e.g., RSV-A-specific neutralizing antibodies, RSV-B-specific neutralizing antibodies, RSV F-protein-specific IgG antibodies, RSV F-protein-specific neutralizing antibodies, and/or antibodies against human metapneumovirus) of about 1-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 12-fold, about 12-fold to about 15-fold, about 15-fold to about 20-fold, about 20-fold to about 25-fold, about 25-fold to about 30-fold, about 30-fold to about 40-fold, about 40-fold to about 50-fold, about 50-fold to about 60-fold, about 60-fold to about 70-fold, about 70-fold to about 80-fold, about 80-fold to about 90-fold, about 90-fold to about 100-fold, or more than about 100-fold compared to baseline. In some embodiments, the methods provided herein result in an increase in antibodies (e.g., in an increase in RSV-A-specific neutralizing antibodies, RSV-B-specific neutralizing antibodies, RSV F-protein-specific IgG antibodies, RSV F-protein-specific neutralizing antibodies, and/or antibodies against human metapneumovirus) of at least about 3-fold, at least about 4-fold, at least about 5-fold, at least about 10-fold, at least about 15-fold, at least about 20-fold, or at least about 25-fold compared to baseline.

By “baseline” is meant a measurement of antibodies immediately prior to administration of the first dose of an immunogenic composition provided herein. In some embodiments, the increase in antibodies (e.g., RSV-A-specific neutralizing antibodies, RSV-B-specific neutralizing antibodies, RSV F-protein-specific IgG antibodies, RSV F-protein-specific neutralizing antibodies, and/or antibodies against human metapneumovirus) compared to baseline is detectable within about 3 days to about 7 days, about 1 week to about 2 weeks, about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 3 months to about 4 months, about 4 months to about 5 months, about 5 months to about 6 months, about 6 months to about 9 months, about 9 months to about 12 months, about 12 months to about 18 months, about 18 months to about 24 months, about 2 years to about 3 years, about 3 years to about 4 years, about 4 years to about 5 years, or about 5 years to about 10 years of administration of the immunogenic composition. In some embodiments, the increase in antibodies (e.g., in an increase in RSV-A-specific neutralizing antibodies, RSV-B-specific neutralizing antibodies, RSV F-protein-specific IgG antibodies, RSV F-protein-specific neutralizing antibodies, and/or antibodies against human metapneumovirus) compared to baseline is detectable within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the immunogenic composition.

In another aspect, the methods provided herein may result in an increase in immune cells in a subject, e.g., an increase in RSV F-protein-specific memory B cells and/or RSV F-protein-specific T cells. The memory B cells and/or T cells may be specific to RSV-A F protein or RSV-B F protein, or they may be reactive to both. The number of immune cells in a subject may be determined using any suitable assay known in the art or described herein, including, without limitation, FACS and flow cytometry.

In some embodiments, the methods provided herein results in an increase in immune cells (e.g., in an increase in RSV F-protein-specific memory B cells and/or RSV F-protein-specific T cells) of about 1-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10-fold, about 10-fold to about 12-fold, about 12-fold to about 15-fold, about 15-fold to about 20-fold, about 20-fold to about 25-fold, about 25-fold to about 30-fold, about 30-fold to about 40-fold, about 40-fold to about 50-fold, about 50-fold to about 60-fold, about 60-fold to about 70-fold, about 70-fold to about 80-fold, about 80-fold to about 90-fold, about 90-fold to about 100-fold, or more than about 100-fold compared to baseline. In some embodiments, the methods provided herein result in an increase in immune cells in a subject, e.g., an increase in RSV F-protein-specific memory B cells and/or RSV F-protein-specific T cells. In some embodiments, the methods provided herein results in an increase in immune cells (e.g., in an increase in RSV F-protein-specific memory B cells and/or RSV F-protein-specific T cells) of at least about 3-fold, at least about 4-fold, at least about 5-fold, at least about 10-fold, at least about 15-fold, at least about 20-fold, or at least about 25-fold compared to baseline. The memory B cells and/or T cells may be specific to RSV-A F protein or RSV-B F protein, or they may be reactive to both.

In some embodiments, the increase in immune cells (e.g., in an increase in RSV F-protein-specific memory B cells and/or RSV F-protein-specific T cells) compared to baseline is detectable within about 3 days to about 7 days, about 1 week to about 2 weeks, about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 3 months to about 4 months, about 4 months to about 5 months, about 5 months to about 6 months, about 6 months to about 9 months, about 9 months to about 12 months, about 12 months to about 18 months, about 18 months to about 24 months, about 2 years to about 3 years, about 3 years to about 4 years, about 4 years to about 5 years, or about 5 years to about 10 years of administration of the immunogenic composition. In some embodiments, the increase in increase in immune cells (e.g., in an increase in RSV F-protein-specific memory B cells and/or RSV F-protein-specific T cells) compared to baseline is detectable within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the immunogenic composition. The memory B cells and/or T cells may be specific to RSV-A F protein or RSV-B F protein, or they may be reactive to both.

As used herein, an “effective amount” refers to an amount of the immunogenic composition that is effective for treating and/or limiting RSV infection (e.g., RSV-A infection and/or RSV-B infection). The polypeptide, virus-like particle, composition, nucleic acid, pharmaceutical composition, or vaccine of any embodiment herein are typically formulated as a pharmaceutical composition, such as those disclosed above, and can be administered via any suitable route, including intranasally, sublingually, orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood).

Dosage regimens can be adjusted to provide the optimum desired response (e.g., a therapeutic or prophylactic response). A suitable dosage range may, for instance, be 0.1 μg/kg to 0.5 μg/kg body weight, 0.5 μg/kg to 1 μg body weight, 1 μg/kg to 2 μg/kg body weight, 2 μg/kg to 3 μg/kg body weight, 3 μg/kg to 4 μg/kg body weight, 4 μg/kg to 5 μg/kg body weight, 5 μg/kg to 6 μg/kg body weight, 6 μg/kg to 7 μg/kg body weight, 7 μg/kg to 8 μg/kg body weight, 8 μg/kg to 9 μg/kg body weight, 9 μg/kg to 10 μg/kg body weight, 10 μg/kg to 15 μg/kg body weight, 15 μg/kg to 20 μg/kg body weight, 20 μg/kg to 25 μg/kg body weight, 25 μg/kg to 30 μg/kg body weight, 30 μg/kg to 35 μg/kg body weight, 35 μg/kg to 40 μg/kg body weight, 40 μg/kg to 45 μg/kg body weight, 45 μg/kg to 50 μg/kg body weight, 50 μg/kg to 55 μg/kg body weight, 55 μg/kg to 60 μg/kg body weight, 60 μg/kg to 65 μg/kg body weight, 65 μg/kg to 70 μg/kg body weight, 70 μg/kg to 75 μg/kg body weight, 75 μg/kg to 80 μg/kg body weight, 80 μg/kg to 85 μg/kg body weight, 85 μg/kg to 90 μg/kg body weight, 90 μg/kg to 95 μg/kg body weight, 95 μg/kg to 100 μg/kg body weight, 100 μg/kg to 150 μg body weight, 150 μg/kg to 200 μg body weight, 200 μg/kg to 250 μg/kg body weight, 250 μg/kg to 300 μg/kg body weight, 300 μg/kg to 350 μg/kg body weight, 350 μg/kg to 400 μg/kg body weight, 400 μg/kg to 450 μg/kg body weight, 450 μg/kg to 500 μg body weight, 500 μg/kg to 550 μg body weight, 550 μg/kg to 600 μg body weight, 600 μg/kg to 650 μg body weight, 650 μg/kg to 700 μg body weight, 700 μg/kg to 750 μg/kg body weight, 750 μg/kg to 800 μg/kg body weight, 800 μg/kg to 850 μg/kg body weight, 850 μg/kg to 900 μg/kg body weight, 900 μg/kg to 950 μg/kg body weight, 950 μg/kg to 1 mg/kg body weight, 1 mg/kg to 2 mg/kg body weight, 2 mg/kg to 3 mg/kg body weight, 3 mg/kg to 4 mg/kg body weight, 4 mg/kg to 5 mg/kg body weight, 5 mg/kg to 6 mg/kg body weight, 6 mg/kg to 7 mg/kg body weight, 7 mg/kg to 8 mg/kg body weight, 8 mg/kg to 90 mg/kg body weight, 90 mg/kg to 100 mg/kg body weight, 100 mg/kg to 150 mg/kg body weight, 150 mg/kg to 200 mg/kg body weight, 200 mg/kg to 250 mg/kg body weight, 250 mg/kg to 300 mg/kg body weight, 300 mg/kg to 350 mg/kg body weight, 350 mg/kg to 400 mg/kg body weight, 400 mg/kg to 450 mg/kg body weight, 450 mg/kg to 500 mg/kg body weight, 500 mg/kg to 550 mg/kg body weight, 550 mg/kg to 600 mg/kg body weight, 600 mg/kg to 650 mg/kg body weight, 650 mg/kg to 700 mg/kg body weight, 700 mg/kg to 750 mg/kg body weight, 750 mg/kg to 800 mg/kg body weight, 800 mg/kg to 850 mg/kg body weight, 850 mg/kg to 900 mg/kg body weight, 900 mg/kg to 950 mg/kg body weight, or 950 mg/kg to 1 g/kg of the polypeptide or virus-like particle thereof.

The composition can be delivered in a single bolus, or may be administered more than once (e.g., 2, 3, 4, 5, or more times) as determined by attending medical personnel. In some embodiments, about 1 μg, about 2 μg, about 3 μg, about 4 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 100 μg, about 125 μg, about 150 μg, about 175 μg, about 200 μg, about 225 μg, about 250 μg, about 275 μg, about 300 μg, about 325 μg, bout 350 μg, about 375 μg, about 400 μg, about 425 μg, about 450 μg, about 475 μg, or about 500 μg of the polypeptide or virus-like particle are administered. In some embodiments, about 5 μg to about 10 μg, about 10 μg to about 15 μg, about 15 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 200 μg, about 200 μg to about 250 μg, about 250 μg to about 300 μg, about 300 μg to about 350 μg, about 350 μg to about 400 μg, about 400 μg to about 450 μg, or about 450 μg to about 500 μg of the polypeptide or virus-like particle are administered.

In some embodiments, about 10 μg to about 100 μg, about 10 μg to about 150 μg, about 10 μg to about 200 μg, about 10 μg to about 250 μg, about 10 μg to about 300 μg, about 10 μg to about 350 μg, about 10 μg to about 400 μg, about 10 μg to about 450 μg, or about 10 μg to about 500 μg of the protein complex are administered.

In some embodiments, about 25 μg to about 100 μg, about 25 μg to about 150 μg, about 25 μg to about 200 μg, about 25 μg to about 250 μg, about 25 μg to about 300 μg, about 25 μg to about 350 μg, about 25 μg to about 400 μg, about 25 μg to about 450 μg, or about 25 μg to about 500 μg of the protein complex are administered.

In some embodiments, about 50 μg to about 100 μg, about 50 μg to about 150 μg, about 50 μg to about 200 μg, about 50 μg to about 250 μg, about 50 μg to about 300 μg, about 50 μg to about 350 μg, about 50 μg to about 400 μg, about 50 μg to about 450 μg, or about 50 μg to about 500 μg of the protein complex are administered.

In some embodiments, about 5 μg to about 150 μg, about 10 μg to about 150 μg, about 25 μg to about 150 μg, about 50 μg to about 150 μg, about 75 μg to about 150 μg, about 100 μg to about 150 μg, or about 125 μg to about 150 μg of the protein complex are administered.

In some embodiments, about 5 μg to about 125 μg, about 10 μg to about 125 μg, about 25 μg to about 125 μg, about 50 μg to about 125 μg, about 75 μg to about 125 μg, or about 100 μg to about 125 μg of the protein complex are administered.

In some embodiments, about 5 μg to about 100 μg, about 10 μg to about 100 μg, about 25 μg to about 100 μg, about 50 μg to about 100 μg, or about 75 μg to about 100 μg of the protein complex are administered.

In some embodiments, about 5 μg to about 75 μg, about 10 μg to about 75 μg, about 25 μg to about 75 μg, or about 50 μg to about 75 μg of the protein complex are administered.

In some embodiments, about 5 μg to about 50 μg, about 10 μg to about 50 μg, or about 25 μg to about 50 μg of the protein complex are administered.

In some embodiments, about 1 μg to about 5 μg, about 5 μg to about 10 μg, about 10 μg to about 15 μg, or about 15 μg to about 25 μg of the protein complex are administered.

In some embodiments, about 1 μg to about 5 μg, about 1 μg to about 10 μg, about 1 μg to about 15 μg, about 1 μg to about 20 μg, about 1 μg to about 25 μg, about 1 μg to about 50 μg, or about 1 μg to about 75 μg of the protein complex are administered.

In some embodiments, 1 μg to 5 μg, 1 μg to 10 μg, 1 μg to 15 μg, 1 μg to 20 μg, 1 μg to 25 μg, 1 μg to 50 μg, or 1 μg to 75 μg of the protein complex are administered.

In some embodiments, about 5 μg to about 10 μg, about 5 μg to about 15 μg, about 5 μg to about 20 μg, about 5 μg to about 25 μg, about 5 μg to about 50 μg, or about 5 μg to about 75 μg of the protein complex are administered.

In some embodiments, 5 μg to 10 μg, 5 μg to 15 μg, 5 μg to 20 μg, 5 μg to 25 μg, 5 μg to 50 μg, or 5 μg to 75 μg of the protein complex are administered.

In some embodiments, about 10 μg to about 15 μg, about 10 μg to about 20 μg, about 10 μg to about 25 μg, about 10 μg to about 50 μg, or about 10 μg to about 75 μg of the protein complex are administered.

In some embodiments, 10 μg to 15 μg, 10 μg to 20 μg, 10 μg to 25 μg, 10 μg to 50 μg, or 10 μg to 75 μg of the protein complex are administered.

In some embodiments, about 25 μg to about 50 μg, or about 25 μg to about 75 μg of the protein complex are administered.

In some embodiments, 25 μg to 50 μg, or 25 μg to 75 μg of the protein complex are administered.

In some embodiments, about 50 μg to about 75 μg of the protein complex are administered.

In some embodiments, 50 μg to 75 μg of the protein complex are administered.

In some embodiments, the unit dose of the pharmaceutical composition comprises at most 1 μg, at most 2.5 μg, at most 5 μg, at most 7.5 μg, at most 10 μg, at most 12.5 μg, at most 15 μg, at most 17.5 μg, at most 20 μg, at most 22.5 μg, or at most 25 μg of the protein complex, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, the methods of the disclosure comprise administering at most 1 μg, at most 2.5 μg, at most 5 μg, at most 7.5 μg, at most 10 μg, at most 12.5 μg, at most 15 μg, at most 17.5 μg, at most 20 μg, at most 22.5 μg, or at most 25 μg of the protein complex, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, the unit dose of the pharmaceutical composition comprises 1 μg, 2.5 μg, 5 μg, 7.5 μg, 10 μg, 12.5 μg, 15 μg, 17.5 μg, 20 μg, 22.5 μg, or 25 μg of the protein complex, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, the methods of the disclosure comprise administering 1 μg, 2.5 μg, 5 μg, 7.5 μg, 10 μg, 12.5 μg, 15 μg, 17.5 μg, 20 μg, 22.5 μg, or 25 μg of the protein complex, where the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, about 1 μg to about 5 μg, about 1 μg to about 10 μg, about 1 μg to about 15 μg, about 1 μg to about 20 μg, about 1 μg to about 25 μg, about 1 μg to about 50 μg, or about 1 μg to about 75 μg of the protein complex are administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 1 μg to 5 μg, 1 μg to 10 μg, 1 μg to 15 μg, 1 μg to 20 μg, 1 μg to 25 μg, 1 μg to 50 μg, or 1 μg to 75 μg of the protein complex are administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, about 5 μg to about 10 μg, about 5 μg to about 15 μg, about 5 μg to about 20 μg, about 5 μg to about 25 μg, about 5 μg to about 50 μg, or about 5 μg to about 75 μg of the protein complex are administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 5 μg to 10 μg, 5 μg to 15 μg, 5 μg to 20 μg, 5 μg to 25 μg, 5 μg to 50 μg, or 5 μg to 75 μg of the protein complex are administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, about 10 μg to about 15 μg, about 10 μg to about 20 μg, about 10 μg to about 25 μg, about 10 μg to about 50 μg, or about 10 μg to about 75 μg of the protein complex are administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 10 μg to 15 μg, 10 μg to 20 μg, 10 μg to 25 μg, 10 μg to 50 μg, or 10 μg to 75 μg of the protein complex are administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 10 μg of the protein complex is administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 25 μg of the protein complex is administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 75 μg of the protein complex is administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 100 μg of the protein complex is administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

In some embodiments, 250 μg of the protein complex is administered, wherein the protein complex comprises 60 copies (20 trimers) of DS-Cav1-I53-50A or DS-Cav1-I53-50AΔcys and 60 copies (12 pentamers) of I53-50B, I53-50B.1, I53-50B.1NegT2 or I53-50B.4PosT1.

Protein complexes and pharmaceutical compositions thereof may be administered on a single dose schedule or a multiple dose schedule. Multiple doses may be used in a primary immunization schedule. In a multiple dose schedule, the various doses may be given by the same or different routes e.g., a parenteral prime and mucosal boost, a mucosal prime and parenteral boost, etc. In some embodiments, the second dose of a multiple dose regimen is administered about 2 weeks, about 3 weeks, about 4 weeks, about 5 weeks, or about 6 weeks after the prior dose. In embodiments, the each subsequent dose is administered 3 weeks after administration of the prior dose. In embodiments, the first dose is administered at day 0, and the second dose is administered at day 21. In embodiments, the first dose is administered at day 0, and the second dose is administered at day 28.

Multiple doses of the boost may be used in a heterologous boost immunization schedule. For example, one or more doses of a primary vaccine may be administered followed by more than one administrations of the boost vaccine. In a multiple dose boost schedule, the various boost doses may be given by the same or different routes e.g., a parenteral prime and mucosal boost, a mucosal prime and parenteral boost, etc. In some embodiments, the second dose of a multiple dose boost regimen is administered about 2 weeks, about 3 weeks, about 4 weeks, about 5 weeks, or about 6 weeks after the prior dose. In some embodiments, each subsequent dose is administered 3 weeks after administration of the prior dose. In some embodiments, the first boost dose is administered at day 0, and the second boost dose is administered at day 21. In some embodiments, the first boost dose is administered at day 0, and the second boost dose is administered at day 28. In some embodiments, the first boost dose is administered at day 0, and the second boost dose is administered at 3 months.

In some embodiments, an immunogenic composition provided herein is administered as a booster of another RSV vaccine, for example, a live-attenuated RSV vaccine, an RSV-A vaccine, and RSV-B vaccine, or a bivalent RSV-A/B vaccine. In some embodiments, the administering comprises administering a first dose and a second dose of the immunogenic composition, wherein the second dose is administered about 2 weeks to about 12 weeks, or about 4 weeks to about 12 weeks after the first dose is administered. In various further embodiments, the second dose is administered about 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the first dose. In another embodiment, three doses may be administered, with a second dose administered about 1, 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the first dose, and the third dose administered about 1, 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the second dose. The second dose may be an RSV booster dose.

In some embodiments, more than two doses of the immunogenic composition are administered. In some embodiments, the first dose and the second dose of the immunogenic composition are administered within about 2, 3, 4, 5, 6, 7, 8, 9, 10, 11, or 12 weeks, about 1 month, about 2 months, about 3 months, about 4 months, about 5 months, about 6 months, about 9 months, or about 12 months of each other, and a third dose is administered about 1 year, about 2 years, about 3 years, about 4 years, or about 5 years after the second dose. In some embodiments, the first dose and the second dose of the immunogenic composition are administered within about 2, 3, 4, 5, 6, 7, 8, 9, 10, 11, or 12 weeks, about 1 month, about 2 months, about 3 months, about 4 months, about 5 months, about 6 months, about 9 months, or about 12 months of each other, and subsequent doses are administered in regular intervals of about 1, 2, 3, 4 or 5 years.

In some embodiments, the subject has previously been infected with RSV (e.g., RSV-A and/or RSV-B). In another embodiment of the methods, the subject is infected with RSV (e.g., RSV-A and/or RSV-B) at the time of being administered a pharmaceutical composition provided herein, wherein the administering elicits an immune response against RSV (e.g., RSV-A and/or RSV-B) in the subject that treats the RSV infection (e.g., RSV-A infection and/or RSV-B infection) in the subject. When the method comprises treating an RSV infection (e.g., RSV-A infection and/or RSV-B infection), the immunogenic compositions are administered to a subject that has already been infected with RSV (e.g., RSV-A and/or RSV-B), and/or who is suffering from symptoms (such as described above) indicating that the subject is likely to have been infected with RSV (e.g., RSV-A and/or RSV-B).

RSV infection (e.g., RSV-A infection and/or RSV-B infection) may be diagnosed using any PCR-based test or antigen-based test known in the art. In some embodiments, the subject has antibodies against RSV. Anti-RSV antibodies (e.g., RSV-A antibodies and/or RSV-B antibodies) may be detected using any serological test known in the art. In some embodiments, the compositions and methods disclosed herein prevent disease following infection with RSV subtypes A and B in older adults.

The protein complexes and pharmaceutical compositions of the disclosure may also be used for heterologous prime-boost vaccination. In some embodiments, a method comprises administering a protein complex or pharmaceutical composition thereof about 2 weeks to about 12 weeks, or about 4 weeks to about 12 weeks after another vaccine, such as a heterologous prime vaccine. In further embodiments, the pharmaceutical composition is administered about 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the other vaccine. In further embodiments, the protein complex or pharmaceutical composition thereof is administered about 2 or more months, about 3 or more months, about 4 or more months, about 5 or more months, about 6 or more months, about 8 or more months, about 10 or more months, or about 12 or months after an earlier vaccine. In some embodiments, a method comprises administering a protein complex or pharmaceutical composition thereof about 2 months to about 8 months, or about 2 months to about 6 months after another vaccine. The interval between first (prime) vaccine and second (boost) vaccine may be 1, 2, 3, 4, 5, 6, 7, 8, 9, 10, 11, or 12 months, or any other suitable interval. The prime vaccine may include multiple doses of the same vaccine, and the heterologous boost vaccine may include multiple doses of the same heterologous vaccine, administered at suitable intervals.

In variations, the method may comprise administering a protein complex or pharmaceutical composition thereof indefinitely, e.g., over regular intervals. For example, the regular intervals may include every 3 months, every 6 months, every 12 months, every 18 months, or every 24 months. In some embodiments, the polypeptide sequence of the antigen may be modified to compensate for antigenic drift.

The protein complexes and pharmaceutical compositions of the disclosure may also be used for homologous prime-boost vaccination (e.g., administering a booster dose following a primary regimen of the same vaccine). In some embodiments, a method comprises administering a protein complex or pharmaceutical composition thereof about 2 weeks to about 12 weeks, or about 4 weeks to about 12 weeks after another vaccine, such as a heterologous prime vaccine. In further embodiments, the pharmaceutical composition is administered about 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the other vaccine. In further embodiments, the protein complex or pharmaceutical composition thereof is administered about 2 or more months, about 3 or more months, about 4 or more months, about 5 or more months, about 6 or more months, about 8 or more months, about 10 or more months, or about 12 or months after an earlier vaccine. In some embodiments, a method comprises administering a protein complex or pharmaceutical composition thereof about 2 months to about 8 months, or about 2 months to about 6 months after another vaccine. The interval between first (prime) vaccine and second (boost) vaccine may be 1, 2, 3, 4, 5, 6, 7, 8, 9, 10, 11, or 12 months, or any other suitable interval. The prime vaccine may include multiple doses of the same vaccine, and the homologous boost vaccine may include multiple doses of the homologous vaccine, administered at suitable intervals. In some embodiments, the method comprises administering a protein complex or pharmaceutical composition thereof continuously, e.g., over regular intervals. For example, the regular intervals may include every 3 months, every 6 months, every 12 months, every 18 months, or every 24 months.

The disclosure further provides prime-boost strategies that employ any known or subsequently developed vaccine—including but not limited to a protein, DNA, mRNA, inactivated virus, or viral vector vaccine—together with a protein complex or pharmaceutical composition as described herein. For example, the protein complexes described herein may be used as a primary vaccine followed by heterologous boost with another vaccine. Optionally, the subject may receive a further vaccination with a protein complex described herein. In other variations, another vaccine is used as the primary vaccine and a protein complex described herein is administered one or more times to boost the response to the primary vaccine.

### Kits

The disclosure further provides kits, which may be used to prepare the virus-like particles and compositions of the disclosure. In some embodiment, a kit provided herein comprises a first component and a second component as disclosed herein, and instructions for use in a method of the disclosure. In some embodiment, a kit comprises one or more unit doses as disclosed herein, and instructions for use in a method of the disclosure. In some embodiments, the kit comprises a vial comprising a single dose of a pharmaceutical composition provided herein. In some embodiments, a kit comprises a vial comprising multiple doses provided herein. In some embodiments, a kit further comprises instructions for use of the pharmaceutical composition. In some embodiments, a kit further comprises a diluent for preparing dilutions of the pharmaceutical composition prior to administration. In some embodiments, the pharmaceutical composition comprises an adjuvant. In some embodiments, a kit comprises a pharmaceutical composition and an adjuvant which must be mixed prior to administration.

### ENUMERATED EMBODIMENTS

The disclosure provides the followed enumerated embodiments:

1. A pharmaceutical composition, comprising a protein complex comprising a first component comprising an RSV F protein and a first multimerization domain; and one or more pharmaceutically acceptable diluents or excipients.

2. The pharmaceutical composition of embodiment 1, wherein the protein complex comprises 2, 3, 4, 5 or more copies of the first component.

3. The pharmaceutical composition of embodiment 1 or embodiment 2, wherein the protein complex comprises a second component comprising a second multimerization domain.

4. The pharmaceutical composition of embodiment 3, wherein the protein complex comprises 2, 3, 4, 5 or more copies of the second component.

5. The pharmaceutical composition of any one of embodiments 1 to 4, wherein the protein complex comprises a third component comprising a third multimerization domain.

6. The pharmaceutical composition of embodiment 5, wherein the protein complex comprises 2, 3, 4, 5 or more copies of the first component.

7. The pharmaceutical composition of any one of embodiments 1 to 6, wherein the protein complex is a nanostructure, nanoparticle, or protein-based virus-like particle.

8. The pharmaceutical composition of any one of embodiments 1 to 7, wherein the components of the protein complex are arranged according to a set of symmetry operators forming dihedral symmetry group.

9. The pharmaceutical composition of any one of embodiments 1 to 8, wherein the components of the protein complex are arranged according to a set of symmetry operators forming cyclic symmetry group.

10. The pharmaceutical composition of any one of embodiments 1 to 9, wherein the protein complex is an icosahedral protein complex.

11. The pharmaceutical composition of any one of embodiments 1 to 9, wherein the protein complex is a tetrahedral protein complex.

12. The pharmaceutical composition of any one of embodiments 1 to 9, wherein the protein complex is an octahedral protein complex.

13. The pharmaceutical composition of embodiments 1 to 12, wherein the first multimerization domain is a trimerization domain and/or the second multimerization domain is a pentamerization domain.

14. The pharmaceutical composition of any one of embodiments 2 to 13, wherein the protein complex comprises 20 copies of the first component and 12 copies of the second component.

15. The pharmaceutical compositions of any one of embodiments 1 to 14, wherein the RSV F protein comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 14, 34, and 35.

16. The pharmaceutical compositions of any one of embodiments 1 to 15, wherein the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 24 and 30-31; and/or wherein the second multimerization domain comprises an amino acid sequence which is at least 95%, at least 96%, at least 97%, at least 98%, at least 99%, or at least 100% identical to an amino acid sequence selected from any one of SEQ ID NOS: 22-23, 25-29, and 32.

17. The pharmaceutical composition of any one of embodiments 1 to 16, wherein the first component comprises an amino acid sequence which is at least 75%, 80%, 85%, 90%, 91%, 92%, 93%, 94%, 95%, 96%, 97%, 98%, 99%, or 100% identical to SEQ ID NO: 6; and

wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 26.

18. The pharmaceutical composition of any one of embodiments 1 to 17, wherein the pharmaceutical composition comprises an oil-in-water adjuvant.

19. The pharmaceutical composition of any one of embodiments 1 to 17, wherein the pharmaceutical composition comprises an aluminum hydroxide-adjuvant.

20. A unit dose of the pharmaceutical composition of any one of embodiments 1 to 19, wherein the unit dose comprises between about 0.5 μg and about 500 μg of the protein complex; between about 0.9 picomoles (pmol) and about 100 pmol of the protein complex; and/or between about 1.8 pmol and about 2,000 pmol of the RSV F protein.

21. The unit dose of embodiment 20, wherein the unit dose comprises between about 25 μg and about 250 μg of the protein complex; between about 5 pmol and about 50 pmol of the protein complex; and/or between about 100 pmol and about 1,000 pmol of the RSV F protein.

22. The unit dose of embodiment 20, wherein the unit dose comprises between about 25 μg and about 75 μg of the protein complex; between about 5 pmol and about 15 pmol of the protein complex; and/or between about 100 pmol and about 250 pmol of the RSV F protein.

23. The unit dose of embodiment 20, wherein the unit dose comprises between about 75 μg and about 250 μg of the protein complex; between about 15 pmol and about 50 pmol of the protein complex; and/or between about 250 pmol and about 1,000 pmol of the RSV F protein.

24. The unit dose of embodiment 20, wherein the unit dose comprises about 25 μg, about 75 μg, or about 250 μg of the protein complex; about 5 pmol, about 15 pmol, or about 50 pmol of the protein complex; and/or about 100 pmol, about 250 pmol, or about 1,000 pmol of the RSV F protein.

25. The unit dose of embodiment 20, wherein the unit dose comprises at least about 25 μg, at least about 75 μg, or at least about 250 μg of the protein complex; at least about 5 pmol, at least about 15 pmol, or at least about 50 pmol of the protein complex; and/or at least about 100 pmol, at least about 250 pmol, or at least about 1,000 pmol of the RSV F protein.

26. The unit dose of embodiment 20, wherein the unit dose comprises at most about 25 μg, at most about 75 μg, or at most about 250 μg of the protein complex; at most about 5 pmol, at most about 15 pmol, or at most about 50 pmol of the protein complex; and/or at most about 100 pmol, at most about 250 pmol, or at most about 1,000 pmol of the RSV F protein.

27. The unit dose of embodiment 20, wherein the unit dose comprises about 1 μg to about 5 μg, about 5 μg to about 10 μg, about 10 μg to about 15 μg, or about 15 μg to about 25 μg of the protein complex.

28. A method of vaccinating a subject, comprising administering to the subject an effective amount of the pharmaceutical composition of any one of embodiments 1 to 19.

29. A method of generating an immune response in a subject, comprising administering to the subject an effective amount of the pharmaceutical composition of any one of embodiments 1 to 19.

30. The method of embodiment 29, wherein the method concurrently generates an immune response to a human metapneumovirus (hMPV) F protein through cross-reactivity with the RSV F protein.

31. A method of treating and/or preventing severe lower respiratory tract infection (LRTI) associated caused by RSV in a subject, comprising administering to the subject an effective amount of the pharmaceutical composition of any one of embodiments 1 to 19.

32. A method of preventing RSV disease in a subject, comprising administering to the subject an effective amount of the pharmaceutical composition of any one of embodiments 1 to 19.

33. The method of any one of embodiments 28 to 32, wherein the subject is at risk of severe RSV disease.

34. The method of embodiment 33, wherein the subject is at risk of severe RSV disease because of underlying diabetes mellitus, cardiovascular disease, or respiratory disease.

35. The method of any one of embodiments 28 to 34, wherein the subject is an adult of over 50 years of age, an adult of over 55 years of age, or an adult of over 60 years of age.

36. The method of any one of embodiments 28 to 34, wherein the subject is an adult at least 50 years of age, an adult at least 55 years of age, or an adult at least 60 years of age.

37. The method of any one of embodiments 28 to 34, wherein the subject is an adult of 18-45 years of age.

38. The method of any one of embodiments 28 to 34, wherein the subject is a healthy adult of 18-45 years of age.

39. The method of any one of embodiments 28 to 34, wherein the subject is over 18 years of age.

40. The method of any one of embodiments 28 to 34, wherein the subject is 18 years of age or greater.

41. The method of any one of embodiments 28 to 34, wherein the subject is 18 years of age or less.

42. A method of generating an immune response in an unborn child, the method comprising administering an effective amount of the pharmaceutical composition of any one of embodiments 1 to 19 to the mother of said unborn child.

43. The method of embodiment 42, wherein the pharmaceutical composition is administered to the mother in the last trimester of the pregnancy.

44. A method of generating an immune response in an infant and/or prevent respiratory syncytial virus (RSV) disease in an infant through maternal immunization of a pregnant subject, the method comprising administering an effective amount of the pharmaceutical composition of any one of embodiments 1 to 19 to the subject.

45. The method of any one of embodiments 28 to 44, wherein the effective amount is between about 0.5 μg and about 500 μg of the protein complex; between about 0.9 picomoles (pmol) and about 100 pmol of the protein complex; and/or between about 1.8 pmol and about 2,000 pmol of the RSV F protein.

46. The method of embodiment 45, wherein the effective amount is between about 25 μg and about 250 μg of the protein complex; between about 5 pmol and about 50 pmol of the protein complex; and/or between about 100 pmol and about 1,000 pmol of the RSV F protein.

47. The method of embodiment 45, wherein the effective amount is between about 25 μg and about 75 μg of the protein complex; between about 5 pmol and about 15 pmol of the protein complex; and/or between about 100 pmol and about 250 pmol of the RSV F protein.

48. The method of embodiment 45, wherein the effective amount is between about 75 μg and about 250 μg of the protein complex; between about 15 pmol and about 50 pmol of the protein complex; and/or between about 250 pmol and about 1,000 pmol of the RSV F protein.

49. The method of embodiment 45, wherein the effective amount is about 25 μg, about 75 μg, or about 250 μg of the protein complex; about 5 pmol, about 15 pmol, or about 50 pmol of the protein complex; and/or about 100 pmol, about 250 pmol, or about 1,000 pmol of the RSV F protein.

50. The method of embodiment 45, wherein the effective amount is at least about 25 μg, at least about 75 μg, or at least about 250 μg of the protein complex; at least about 5 pmol, at least about 15 pmol, or at least about 50 pmol of the protein complex; and/or at least about 100 pmol, at least about 250 pmol, or at least about 1,000 pmol of the RSV F protein.

51. The method of embodiment 45, wherein the effective amount is at most about 25 μg, at most about 75 μg, or at most about 250 μg of the protein complex; at most about 5 pmol, at most about 15 pmol, or at most about 50 pmol of the protein complex; and/or at most about 100 pmol, at most about 250 pmol, or at most about 1,000 pmol of the RSV F protein.

52. The method of embodiment 45, wherein the effective amount is about 0.5 μg to about 1 μg, about 20 μg to about 25 μg, about 70 μg to about 75 μg, about 100 μg to about 125 μg, or about 200 μg to about 250 μg of the protein complex.

53. The method of any one of embodiments 28 to 52, further comprising administering a second dose of the pharmaceutical composition.

54. The method of embodiment 53, wherein the second dose is administered within about 2, 3, 4, 5, 6, 7, 8, 9, 10, 11, or 12 weeks, about 1 month, about 2 months, about 3 months, about 4 months, about 5 months, about 6 months, about 9 months, about 12 months, about 24 months, or about 36 months of the first dose.

55. The method of embodiment 53 or embodiment 54, further comprising administering a third dose of the pharmaceutical composition.

56. The method of embodiments 55, wherein the third dose is administered about 1 year, about 2 years, about 3 years, about 4 years, about 5 years, about 6 years, about 7 years, about 8 years, about 9 years, or about 10 years after the second dose.

57. The method of embodiment 55 or embodiment 56, further comprising administering subsequent doses at regular intervals of about 1, 2, 3, 4 5, 6, 7, 8, 9, or 10 years.

58. The method of any one of embodiments 28 to 57, wherein the method limits the development of an RSV infection in a subject.

59. The method of any one of embodiments 28 to 57, wherein the method limits the development of more severe lower respiratory tract infection (LRTI) in the subject.

60. The method of any one of embodiments 28 to 57, wherein the method results in the production of RSV-A-specific neutralizing antibodies in the subject.

61. The method of embodiment 60, wherein the method results in an increase in RSV-A-specific neutralizing antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

62. The method of embodiment 60 or embodiment 61, wherein the increase in RSV-A-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

63. The method of any one of embodiments 28 to 62, wherein the method results in the production of RSV-B-specific neutralizing antibodies in the subject.

64. The method of embodiment 63, wherein the method results in an increase in RSV-B-specific neutralizing antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

65. The method of embodiment 63 or embodiment 64, wherein the increase in RSV-B-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

66. The method of any one of embodiments 28 to 65, wherein the method results in the production of RSV F-protein-specific IgG antibodies in the subject.

67. The method of embodiment 66, wherein the method results in an increase of RSV F-protein-specific IgG antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

68. The method of embodiment 66 or embodiment 67, wherein the increase in RSV F-protein-specific IgG antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

69. The method of any one of embodiments 58 to 68, wherein the method results in the production of Core-VLP-specific IgG antibodies in the subject.

70. The method of embodiment 69, wherein the method results in an increase in Core-VLP-specific IgG antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

71. The method of embodiment 69 or embodiment 70, wherein the increase in Core-VLP-specific IgG antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

72. The method of any one of embodiments 58 to 68, wherein the method results in the production of substantially no Core-VLP-specific IgG antibodies in the subject.

73. The method of any one of embodiments 58 to 72, wherein the method results in the production of RSV F-protein-specific memory-B-cells in the subject.

74. The method of embodiment 73, wherein the method results in an increase in RSV F-protein-specific memory-B-cells in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

75. The method of embodiment 73 or embodiment 74, wherein the increase in RSV F-protein-specific memory-B-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

76. The method of any one of embodiments 58 to 75, wherein the method results in the production of RSV F-protein-specific T-cells in the subject.

77. The method of embodiment 76, wherein the method results in an increase in RSV F-protein-specific T-cells in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

78. The method of embodiment 76 or embodiment 77, wherein the increase in RSV F-protein-specific T-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

79. The method of any one of embodiments 58 to 78, wherein the method results in the production of neutralizing antibodies against human metapneumovirus in the subject.

80. The method of embodiment 79, wherein the method results in an increase in neutralizing antibodies against human metapneumovirus in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

81. The method of embodiment 79 or embodiment 80, wherein the increase in antibodies against human metapneumovirus is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

82. The method of any one of embodiments 28 to 81, wherein the method prevents severe LRTI associated with or caused by RSV more effectively than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

83. The method of any one of embodiments 28 to 82, wherein the method prevents severe LRTI associated with or caused by RSV more effectively than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

84. The method of any one of embodiments 28 to 83, wherein the method results in greater RSV-A and/or RSV-B-specific neutralizing antibodies than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

85. The method of any one of embodiments 28 to 84, wherein the method results in protective immunity for a longer time period than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

86. The method of any one of embodiments 28 to 85, wherein the method results in a neutralizing antibody response lasting at least 12 months.

87. The method of any one of embodiments 28 to 86, wherein the method results in a protective immunity lasting at least 12 months.

88. The method of any one of embodiments 28 to 87, wherein the adjuvant, if present, increases the durability of the neutralizing antibody response, the cross-protection of the neutralizing antibody response, and/or the magnitude of the B cell or T cell activation in the subject.

89. The method of any one of embodiments 28 to 88, wherein the method causes fewer adverse events than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

90. The method of any one of embodiments 28 to 89, wherein the method results in RSV-A and/or RSV-B specific neutralizing antibodies in the subject at geometric mean titer (GMT) of greater than 1,000 international units per milliliter (IU/mL), greater than 2,000 IU/mL, greater than 3,000 IU/mL, greater than 4,000 IU/mL, greater than 5,000 IU/mL, greater than 6,000 IU/mL, greater than 7,000 IU/mL, greater than 8,000 IU/mL, greater than 9,000 IU/mL, or greater than 10,000 IU/mL.

91. The method of any one of embodiments 28 to 89, wherein the method results in RSV-A and/or RSV-B specific neutralizing antibodies in the subject at GMT of greater than 1,000 IU/mL, greater than 2,000 IU/mL, greater than 3,000 IU/mL, greater than 4,000 IU/mL, greater than 5,000 IU/mL, greater than 6,000 IU/mL, greater than 7,000 IU/mL, greater than 8,000 IU/mL, greater than 9,000 IU/mL, or greater than 10,000 IU/mL, wherein the subject is an adult of age 18 to 45.

92. The method of any one of embodiments 28 to 89, wherein the method results in RSV-A and/or RSV-B specific neutralizing antibodies in the subject at GMT of greater than 1,000 IU/mL, greater than 2,000 IU/mL, greater than 3,000 IU/mL, greater than 4,000 IU/mL, greater than 5,000 IU/mL, greater than 6,000 IU/mL, greater than 7,000 IU/mL, greater than 8,000 IU/mL, greater than 9,000 IU/mL, or greater than 10,000 IU/mL, wherein the subject is an adult of age 60 to 75.

93. The method of any one of embodiments 28 to 89, wherein the method results in RSV-A specific neutralizing antibodies in the subject at GMT of greater than 3,000 IU/mL.

94. The method of any one of embodiments 28 to 89, wherein the method results in RSV-A specific neutralizing antibodies in the subject at GMT of greater than 3,000 IU/mL, wherein the subject is an adult of age 18 to 45.

95. The method of any one of embodiments 28 to 89, wherein the method results in RSV-A specific neutralizing antibodies in the subject at GMT of greater than 3,000 IU/mL, wherein the subject is an adult of age 60 to 75.

96. The method of any one of embodiments 28 to 95, wherein the method results in prefusion RSV F binding IgG antibodies in the subject at GMT of greater than 1,000 immunosorbent assay unit per milliliter (EU/mL), greater than 2,000 EU/mL, greater than 3,000 EU/mL, greater than 4,000 EU/mL, greater than 5,000 EU/mL, greater than 6,000 EU/mL, greater than 7,000 EU/mL, greater than 8,000 EU/mL, greater than 9,000 EU/mL, or greater than 10,000 EU/mL.

97. The method of any one of embodiments 28 to 96, wherein the method results in prefusion RSV F binding IgG antibodies in the subject at GMT of greater than 1,000 EU/mL, greater than 2,000 EU/mL, greater than 3,000 EU/mL, greater than 4,000 EU/mL, greater than 5,000 EU/mL, greater than 6,000 EU/mL, greater than 7,000 EU/mL, greater than 8,000 EU/mL, greater than 9,000 EU/mL, or greater than 10,000 EU/mL, wherein the subject is an adult of age 18 to 45.

98. The method of any one of embodiments 28 to 97, wherein the method results in prefusion RSV F binding IgG antibodies in the subject at GMT of greater than 1,000 EU/mL, greater than 2,000 EU/mL, greater than 3,000 EU/mL, greater than 4,000 EU/mL, greater than 5,000 EU/mL, greater than 6,000 EU/mL, greater than 7,000 EU/mL, greater than 8,000 EU/mL, greater than 9,000 EU/mL, or greater than 10,000 EU/mL, wherein the subject is an adult of age 60 to 75.

99. The method of any one of embodiments 28 to 98, wherein the method results in RSV-A specific neutralizing antibodies in the subject at GMT of greater than 3,000 EU/mL.

100. The method of any one of embodiments 28 to 99, wherein the method results in RSV-A specific neutralizing antibodies in the subject at GMT of greater than 3,000 EU/mL, wherein the subject is an adult of age 18 to 45.

101. The method of any one of embodiments 28 to 100, wherein the method results in RSV-A specific neutralizing antibodies in the subject at GMT of greater than 3,000 EU/mL, wherein the subject is an adult of age 60 to 75.

102. The method of any one of embodiment 28 to 101, wherein the method results in an RSV-A seroresponse rate at 4-fold rise or 8-fold rise of at least 20%, at least 30%, at least 40%, at least 50%, at least 60%, at least 70%, at least 80% or at least 90%.

103. The method of any one of embodiment 28 to 102, wherein the method results in an RSV-A seroresponse rate at 4-fold rise or 8-fold rise of 20% to 40%, 30% to 50%, 40% to 60%, 50% to 70%, 60% to 80%, or 70% to 90%.

104. The method of any one of embodiment 28 to 103, wherein the method results in an RSV-B seroresponse rate at 4-fold rise or 8-fold rise of at least 20%, at least 30%, at least 40%, at least 50%, at least 60%, at least 70%, at least 80% or at least 90%.

105. The method of any one of embodiment 28 to 104, wherein the method results in an RSV-B seroresponse rate at 4-fold rise or 8-fold rise of 20% to 40%, 30% to 50%, 40% to 60%, 50% to 70%, 60% to 80%, or 70% to 90%.

106. The method of any one of embodiments 28 to 105, wherein the method results in serious adverse events in fewer than 20%, fewer than 10%, fewer than 5%, or about 0% of subjects administered the pharmaceutical composition.

107. The method of any one of embodiments 28 to 105, wherein the method results in serious adverse events in fewer than 20%, fewer than 10%, fewer than 5%, or about 0% of subjects administered the pharmaceutical composition, wherein the subjects are adults of age 18 to 45.

108. The method of any one of embodiments 28 to 105, wherein the method results in serious adverse events in fewer than 20%, fewer than 10%, fewer than 5%, or about 0% of subjects administered the pharmaceutical composition, wherein the subjects are adults of age 60 to 75.

109. The method of any one of embodiments 28 to 105, wherein the method results in serious adverse events in fewer than 20%, fewer than 10%, fewer than 5%, or about 0% of subjects administered the pharmaceutical composition.

110. The method of any one of embodiments 28 to 105, wherein the method results in serious adverse events in fewer than 20%, fewer than 10%, fewer than 5%, or about 0% of subjects administered the pharmaceutical composition, wherein the subjects are adults of age 18 to 45.

111. The method of any one of embodiments 28 to 105, wherein the method results in serious adverse events in fewer than 20%, fewer than 10%, fewer than 5%, or about 0% of subjects administered the pharmaceutical composition, wherein the subjects are adults of age 60 to 75.

112. The method of any one of embodiments 28 to 111, wherein the pharmaceutical composition is substantially free of any adjuvant.

113. The method of any one of embodiments 28 to 112, wherein the pharmaceutical composition is substantially free of aluminum salt adjuvants.

114. The method of any one of embodiments 28 to 112, wherein the pharmaceutical composition is substantially free of alum.

### EXAMPLES

The following non-limiting examples are provided to illustrate operation of the embodiments disclosed herein. All Examples were performed using the non-limiting embodiment of a protein-based VLP, an icosahedral structure formed from a first component (SEQ ID NO: 6) displaying RSV F DS-Cav1 (SEQ ID NO: 14) on a first multimerization domain (SEQ ID NO: 24); complexed with a second component (SEQ ID NO: 26), having 20 copies of the first component and 12 copies of the second component.

### Example 1: Characterization of Immune Responses Induced by an RSV Vaccine in Naïve Mice

The purpose of this study was to evaluate the ability of an RSV vaccine with and without aluminum hydroxide adjuvant to generate RSV neutralizing antibody response in naïve Balb/c mice.

### Methods:

The study included seventy female Balb/c mice, distributed into 7 groups of 10 animals per group. The candidate RSV vaccine was administered intramuscularly at three dose levels (8.33 μg, 2.5 μg and 0.83 μg) either unadjuvanted, adjuvanted with Alhydrogel (aluminum hydroxide adjuvant), or adjuvanted with Addavax (oil-in-water emulsion) on Day 0, Day 21 and Day 42. Serum samples were obtained on Days 0, 42 and 56 to measure neutralization antibody titers using a virus neutralization assay. Clinical observations were made daily and animals were weighed weekly. The study included seventy female Balb/c mice, distributed into 7 groups of 10 animals per group (See Table 2 below).

TABLE 2

| Group No. | Experimental | Antigen | | |
|---|---|---|---|---|
| (N = 10 mice) | Group | per Dose | | Adjuvant |
| 1 | Low | 8.3 | mcg | N/A |
| 2 | Low alum | 8.3 | mcg | Alhydrogel |
| 3 | Medium | 2.5 | mcg | N/A |
| 4 | Medium alum | 2.5 | mcg | Alhydrogel |
| 5 | High | 0.83 | mcg | N/A |
| 6 | High alum | 0.83 | mcg | Alhydrogel |
| 7 | High oil in water | 8.3 | mcg | Addavax |

| | | | |
|---|---|---|---|
| 8 | Placebo | N/A | N/A |

### Results

Clinical observations were normal following vaccination with all doses and formulations of the RSV vaccine. All mice remained healthy and survived to study termination.

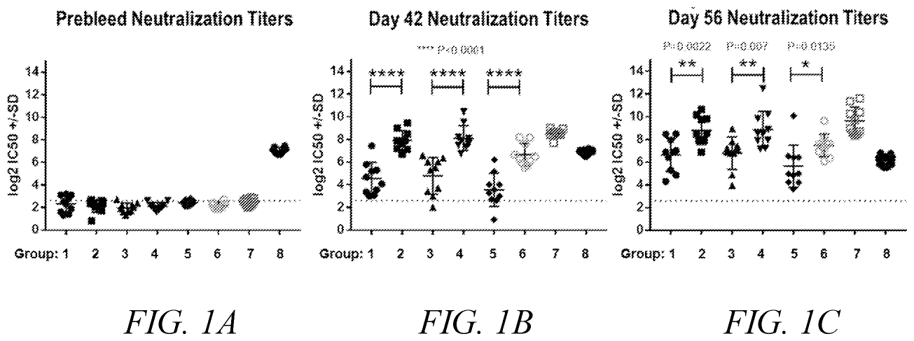

RSV neutralization titers on Days 0, 42 (post booster Dose 1) and 56 (post booster Dose 2) were statistically higher for the adjuvanted groups as compared to the corresponding non-adjuvanted group (FIGS. 1A-1C).

### Conclusions

The results from the virus neutralization indicated that after a priming dose on Day 0 and one or two booster doses (on Day 21 and Day 42, respectively), formulations with aluminum hydroxide adjuvant (Alhydrogel) enhanced the immunogenicity of the RSV candidate vaccine, compared to the aqueous formulations. The adjuvant effect of aluminum hydroxide was demonstrated at all dose levels tested (8.33 μg, 2.5 μg and 0.83 μg).

### Example 2: Characterization of Immune Responses Induced by an RSV Vaccine in Primed (Seropositive) Mice

The purpose of this study was to evaluate the ability of an RSV vaccine with and without aluminum hydroxide adjuvant to boost RSV-neutralizing antibody responses and cellular immune responses in a seropositive RSVA2 Balb/c mouse model.

### Methods

Two hundred female Balb/c mice, 6-8 weeks of age, were infected with 1×10⁶ pfu RSV A2 intranasally and housed for twelve weeks to allow for the resolution of the infection and the establishment of immunological memory. Twenty mice were not infected and served as naïve controls.

Animals were allocated to 19 experimental groups according to neutralizing antibody titers from Day 28 serum samples to obtain comparable groups. The mice were vaccinated intramuscularly on Day 91 with one of four different dose levels of the candidate RSV vaccine (1.66, 0.5, 0.16 and 0.016 μg) or an equivalent amount of a stabilized RSV F-protein (1, 0.3, 0.1, and 0.01 μg) (Table 3). RSV neutralization titers were measured on Days 0, 28, 87, and 101. Serum samples were collected from each animal prior to infection on Day 0, Day 28, Day 87 and Day 101 (Table 4). Splenocytes were isolated from a subset of animals on Days 101 and 102 and stimulated with RSV-specific peptides in an ELISpot assay or a cytokine release assay (a cell-mediated immunity assay).

TABLE 3
Treatment Groups

| Group No. | RSVA2 | Experimental | Antigen | | |
|---|---|---|---|---|---|
| (N = 9-10 mice) | on Day 0 | Group | per Dose | | Adjuvant |
| 1 | + | Saline | 0 | | None |
| 2 | + | RSV Vaccine | 1.66 | μg | None |
| 3 | + | RSV Vaccine | 1.66 | μg | Alhydrogel |
| 4 | + | RSV Vaccine | 0.5 | μg | None |
| 5 | + | RSV Vaccine | 0.5 | μg | Alhydrogel |
| 6 | + | RSV Vaccine | 0.16 | μg | None |
| 7 | + | RSV Vaccine | 0.16 | μg | Alhydrogel |
| 8 | + | RSV Vaccine | 0.016 | μg | None |
| 9 | + | RSV Vaccine | 0.016 | μg | Alhydrogel |
| 10 | + | RSV F-protein | 1 | μg | None |
| 11 | + | RSV F-protein | 1 | μg | Alhydrogel |
| 12 | + | RSV F-protein | 0.3 | μg | None |
| 13 | + | RSV F-protein | 0.3 | μg | Alhydrogel |
| 14 | + | RSV F-protein | 0.1 | μg | None |
| 15 | + | RSV F-protein | 0.1 | μg | Alhydrogel |
| 16 | + | RSV F-protein | 0.01 | μg | None |
| 17 | + | RSV F-protein | 0.01 | μg | Alhydrogel |

| | | | | |
|---|---|---|---|---|
| 18 | + | RSV | 10exp 6 | None |
| 19 | − | Saline | 0 | None |

TABLE 4
Study Design

| | Infect | | Bleed | Splenocyte |
|---|---|---|---|---|
| | (intranasal | | (neutralization | collection |
| Day | route) | Immunize | antibody titer) | (CMI) |
| 0 | X | | X | |
| 28 | | | X | |
| 61 | | | X | |
| 87 | | | X | |
| 91 | | X | | |
| 101 | | | X | X |
| 102 | | | | X |

### Results

Clinical observations were normal following vaccination with all of the test vaccines. All mice remained healthy and survived to study termination.

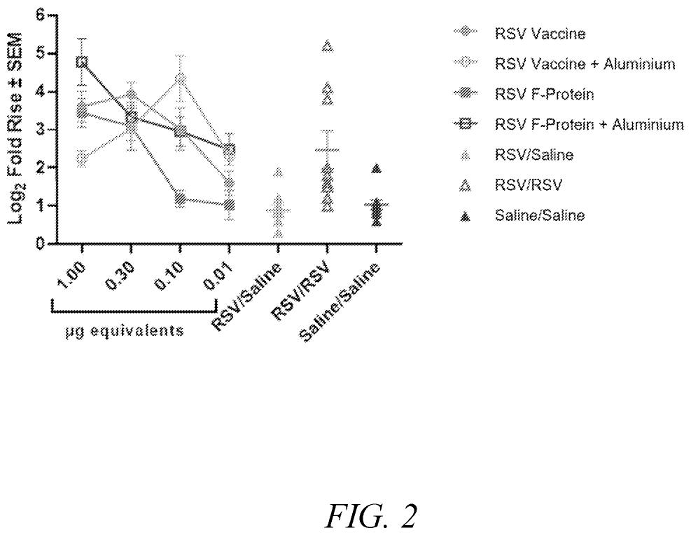

The results from the virus neutralization assay demonstrated that neutralizing titers were increased following vaccination of RSV-primed mice with the RSV vaccine formulated with and without Alhydrogel (FIG. 2).

Low doses of RSV vaccine induced superior neutralizing titers compared to RSV F-protein (0.3, 0.1 and 0.01 μg antigen normalized as RSV F-protein equivalent) in the absence of Alhydrogel.

Similar responses were observed with RSV vaccine and RSV F-protein in saline at the 1 μg dose, likely due to the saturation of the humoral response. Unexpectedly, 1 μg RSV F-protein/Alhydrogel showed a higher neutralizing titer than an equivalent dose of RSV vaccine/Alhydrogel.

No consistent increase in titers could be demonstrated for the RSV vaccine formulated with Alhydrogel compared to saline formulated RSV vaccine.

An increase of secreted cytokines was also measured with a profile (higher Th1 cytokines IFNγ and TNFα than Th2 cytokines IL-4, IL-5 and IL-13) similar to the response observed after RSV reinfection (data not shown).

### Conclusions

The results from the virus neutralization assay demonstrated that in RSV-primed mice, an increase of neutralizing antibody titers was observed following vaccination with an RSV vaccine with and without Alhydrogel for all the doses tested. In contrast to what was observed in naïve mice, no consistent increase in neutralizing antibody titers could be demonstrated for the vaccine formulated with Alhydrogel compared to the vaccine formulated with saline.

The results from the ELISpot analysis on spleen cells indicated that IFNγ-producing CD4+ cells, and to a lesser extent CD8+ T cells specific for F peptides, can be boosted by the vaccine formulated with or without Alhydrogel.

In conclusion, the RSV vaccine tested with and without Alhydrogel was able to boost RSV neutralizing antibody titers and cellular immune responses in a seropositive RSVA2 Balb/c mouse model.

### Example 3: Assessment of the Safety and Immunogenicity in Rabbits

This study was designed to explore the safety and immunogenicity of up to 3 vaccinations of soluble RSV vaccine (in Addavax, oil-in-water emulsion) and aluminum hydroxide (Alhydrogel)-adjuvanted formulations in rabbits. The purpose of this study was to develop an initial assessment of the safety of and RSV vaccine in rabbits; to assess the immune response in vaccinated animals; and to assess whether the presence of pre-existing antibodies to the VLP core (i.e., the protein complex lacking the RSV F-protein) interferes with the response to the RSV vaccine in rabbits.

### Methods

Five New Zealand White (NZW) female rabbits (Group 1) were administered 0.5 mg of VLP core with Addavax by IM injection on Days 1 and 14. Subsequently, the rabbits in Group 1 were vaccinated with 0.25 mg RSV vaccine adsorbed to 0.5 mg aluminum hydroxide adjuvant on Days 56, 70 and 84. Two additional groups of 3 female rabbits each were vaccinated on Days 56, 70 and 84 with 0.25 mg RSV vaccine alone (Group 2) or 0.25 mg adsorbed to 0.5 mg aluminum hydroxide adjuvant (Group 3) with no VLP core prior administration. All vaccinations consisted of a total volume of 0.5 mL split into two 0.25 mL injections (injection on the right and left thigh).

Animals were monitored daily for morbidity and/or mortality throughout the study, and body weights were collected prior to and 24 hours following each vaccination and on Day 28 for all groups. Clinical chemistry and hematology were evaluated pretreatment (Day 0) and one day after the first vaccination (Day 57) for Groups 2 and 3. Sera was collected for RSV neutralizing antibody titers prior to the first vaccination (Day 0) for all groups and on Days 13, 27, 55, 69, 83 and 98 for Group 1 and on Days 0, 55, 57, 69, 82 and 98 for Groups 2 and 3. All animals were sacrificed 14 days after the last administration (Day 98), and gross necropsy was performed on Group 2 and Group 3 animals.

### Results

The animals gained weight throughout the study with no abnormal clinical signs and were alert and healthy until their intended sacrifice. Additionally, there were no changes in clinical chemistry parameters attributed to soluble or aluminum hydroxide-adjuvanted RSV vaccine. The only potential test article-related changes were limited to increases in blood basophils and eosinophils following the first vaccination, compared to pre-vaccination values. Upon sacrifice on Day 98, no remarkable gross findings were noted on any organ during necropsy.

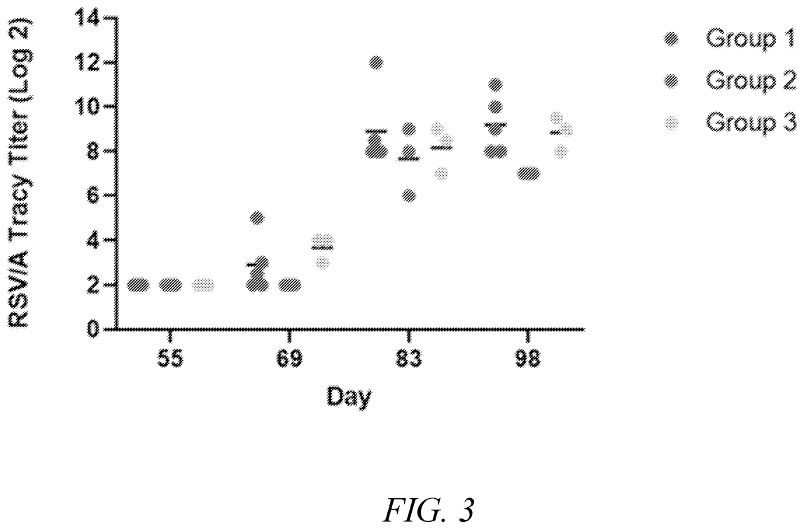

Most of the animals (6 of 8 in Groups 1 and 3) that received the aluminum hydroxide-adjuvanted formulation of RSV vaccine generated a measurable neutralizing response after a single vaccination (Day 69) (FIG. 3). Neutralizing antibody titers were higher after 2 vaccinations of RSV vaccine (Day 83) compared to pre-immune sera (Day 55) (FIG. 3). A third vaccination (Day 98) did not noticeably increase neutralization titers (FIG. 3). Neutralizing antibody titers in sera from animals pre-vaccinated with the first component of RSV vaccine on Days 1 and 14 (Group 1) were similar to those in naïve animals (Group 3) following two or three vaccinations (Day 83 and 98, respectively).

### Conclusions

The maximum anticipated human dose of 250 μg of RSV vaccine, administered either as an aqueous antigen or adsorbed to aluminum hydroxide adjuvant, was well-tolerated by the rabbits. Following two administrations of the test articles a functional immune response (i.e., RSV/A neutralizing antibodies) was induced in all animals, confirming the immunogenicity of the vaccine. Prior vaccination with the VLP core lacking the RSV F antigen induced antibodies which did not appear to affect the ability the RSV vaccine formulated with aluminum hydroxide adjuvant to generate neutralizing antibodies.

### Example 4: Repeat Intramuscular Dose Vaccine Toxicity Study in Rabbits with a 4-Week Recovery

The purpose of this study was to evaluate in NZW rabbits the toxicity of multiple injections of an RSV vaccine with or without aluminum hydroxide adjuvant.

### Methods

NZW rabbits were vaccinated (0.5 mL/dose) with sterile saline (negative control), 0.25 mg RSV vaccine alone or 0.25 mg RSV vaccine adsorbed to 0.5 mg aluminum hydroxide adjuvant once every 2 weeks for a total of three vaccinations (i.e., administrations on Day 1, 15 and 29). The volume per injection and the dose level of RSV vaccine without adjuvant and with aluminum hydroxide adjuvant are equivalent to the intended clinical dose and injection volume. Three days following the third vaccination, 5 animals/sex were sacrificed (on Day 32); the remaining animals (5/sex/group) were maintained for an additional 4 weeks and sacrificed on recovery day (RD) 29 (Study Day 61). See Table 5 for group allocation.

The formulation of RSV vaccine contained 0.5 mg/mL RSV vaccine in 20 mM Tris, 200 mM NaCl, 4% sucrose, pH 7.8±0.2 with or without 1 mg/mL aluminum hydroxide adjuvant.

TABLE 5
Group Allocation for Study

| | | Aluminum | | | |
|---|---|---|---|---|---|
| | | hydroxide | Number of animals | | |
| | Vaccine | adjuvant | | Terminal | Recovery |
| Group | (mg/dose) | (mg/dose) | Total | sacrifice | sacrifice |
| 1 (saline) | 0 | 0 | 10M/10F | 5M/5F | 5M/5F |
| 2 (without | 0.25 | 0 | 10M/10F | 5M/5F | 5M/5F |
| adjuvant) | | | | | |
| 3 (with | 0.25 | 0.5 | 10M/10F | 5M/5F | 5M/5F |
| aluminum | | | | | |
| hydroxide | | | | | |
| adjuvant) | | | | | |

Viability checks for morbidity and mortality were performed twice daily, and cage-side observations were conducted once daily during the dosing and recovery phases. Detailed clinical observations were recorded once weekly and prior to sacrifice in the dosing phase and once weekly during recovery. Body weights were obtained prior to and 24 hours after each vaccination and on Days 8 and 31 during the dosing phase and once weekly during recovery, and food consumption was recorded daily throughout the study. The injection site was observed using a modified Draize technique prior to dosing on Day 1 and approximately 1, 4, 24, 48 and 72 hours after each vaccination. Body temperatures were recorded prior to each administration and approximately 6 and 24 hours post-dose. Eye exams were conducted pretest and the last week of the dosing and recovery period. Clinical chemistry, hematology, coagulation and C-reactive protein were evaluated pretest, approximately 48 hours after the first and last administration (on Days 3 and 41).

For immunogenicity analysis, serum samples were collected pretest, prior to dosing on Day 29 and on RD29. RSV neutralizing antibody titers were measured.

At scheduled sacrifice (Day 31 and RD29), a complete necropsy (gross pathology) was performed, a subset of organs was weighed, a bone marrow smear was collected, and a complete panel of tissues was microscopically examined.

### Results

All animals survived until their scheduled sacrifice days with no test article effects noted for clinical or dermal observations, ophthalmic exams, body weights, food consumption, body temperature, gross pathology or hematology, coagulation or clinical chemistry parameters. The only test article-related effects were observed at terminal sacrifice (3 days following the last administration). These findings consisted of a minimal increase in lymphocytes in the spleen, characterized by multifocal expanded germinal centers in the white pulp, with varying expansion of the marginal zone, with a general correlative minor increase in mean absolute and relative spleen weights up to approximately 1.3 fold in both male and female animals compared to saline. Four weeks after the last administration, splenic lymphocyte numbers were still high in the females of both groups but at a lower incidence, suggesting reversibility, whereas in the males the effects fully resolved. Spleen organ weights were similar to Group 1 (saline) weights, demonstrating complete recovery in both males and females. In general, these findings were considered nonadverse as they were minor in nature and consistent with an immune response against the test articles.

Following repeated IM injection of 0.25 mg/dose RSV vaccine, RSV/A-specific neutralizing antibodies were elicited. Peak serum neutralizing antibody titers were detected by Day 29 of the dosing phase (prior to the last administration). Four weeks following the last vaccine injection, a slight decrease in RSV/A-specific neutralizing antibody titers was observed. Animals that had been administered RSV vaccine with aluminum hydroxide adjuvant (Group 3) generated approximately 3.4- and 5.1-fold higher titers on Day 29 of the dosing phase and at the end of the recovery phase, respectively, compared to animals that had been administered RSV vaccine without adjuvant (Group 2).

### Conclusions

Repeat IM injection once every 2 weeks (Days 1, 15, and 29) of 0.25 mg RSV vaccine was immunogenic and well-tolerated by male and female rabbits. Peak RSV/A-specific neutralizing antibody titers occurred by Day 29 of the dosing phase (prior to the third administration), with a small decrease in titers following the 4-week recovery period. Animals that had been administered RSV vaccine with aluminum hydroxide adjuvant generated approximately 3.4- and 5.1-fold higher titers on Day 29 of the dosing phase and at the end of the recovery period, respectively, compared to animals that had been administered RSV vaccine without adjuvant. Similar test article-related findings were observed in animals administered both RSV vaccine without adjuvant and RSV vaccine with aluminum hydroxide adjuvant, consisting of an increase of lymphocytes present in the spleen with correlated increased spleen weights at the terminal sacrifice (3 days following the last administration). At the recovery sacrifice (4 weeks after the last administration), minimally-increased lymphocytes persisted in the spleen of some females though at a lower incidence, suggesting partial reversibility, and was completely reversed in the males. These findings, which were minor in nature, are consistent with an immune response against the test article and were considered nonadverse.

### Example 5: A Phase 1/1b (Ph1/1b) Study to Evaluate the Safety and Immunogenicity of an RSV Vaccine in Healthy Adults

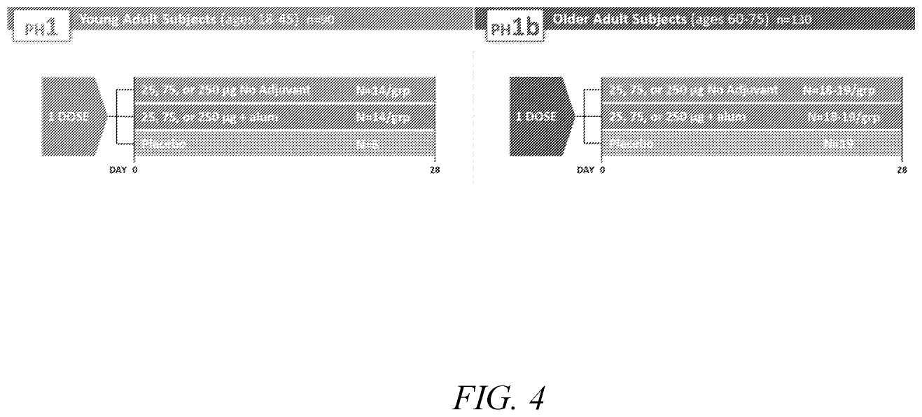

This example describes a Phase 1/1b Study is a randomized placebo-controlled observer-blind study to assess the safety and immunogenicity of a single intramuscular (IM) dose of RSV vaccine as aqueous or aluminum hydroxide-adjuvanted formulations. The trial design is shows in FIG. 4. A total of six formulations (three dose levels of the aqueous formulation and three dose levels of the aluminum hydroxide-adjuvanted formulation) were tested. Placebo was administered as a control. The study was conducted in two parts:
Part 1: Phase 1 First In Human (FIH) assessment in healthy young adults aged 18-45 years (N=90).
Part 2: Phase 1b evaluation in healthy older adults aged 60-75 years (N=217).

The subject allocation in each Part to the seven study arms (6 formulations and placebo) is given in Table 6.

TABLE 6

| | | Aluminum | Part 1 | Part 2 | Overall |
|---|---|---|---|---|---|
| Study | VLP | hydroxide | N = 90 | N = 130 | N = 220 |
| Arm | (μg) | (μg) | 18-45 y | 60-75 y | 18-75 y |
| A | 25 | 0 | 14 | 31 | 45 |
| B | 25 | 500 | 14 | 31 | 45 |
| C | 75 | 0 | 14 | 31 | 45 |
| D | 75 | 500 | 14 | 31 | 45 |
| E | 250 | 0 | 14 | 31 | 45 |
| F | 250 | 500 | 14 | 31 | 45 |
| G | 0 (placebo) | 0 | 6 | 31 | 37 |

The duration of subject participation in the study will be approximately 6 months. The three dose levels tested in the study, 25 μg, 75 μg, and 250 μg of the VLP protein complex, are equivalent in mass to 14 μg, 42 μg, and 140 μg, respectively, of a soluble RSV F antigen-based on subtracting the mass of the VLP core from the mass of the complete VLP including both displayed antigens and its core.

### Objectives

### Primary Objective:

To select for further clinical development the optimal formulation of the RSV vaccine, in terms of VLP quantity and the requirement of aluminum hydroxide as an adjuvant, by assessing in healthy young- (18-45 years) and healthy older-adult (60-75 years) populations:
Serious adverse events (SAEs), medically attended adverse events (MAAEs) and adverse events (AEs) leading to study withdrawal;
Day-28 seroresponse rate (SRR) (percentage of subjects with ≥4-fold rise in titer versus baseline (Day 0)) and geometric mean fold rise (GMFR) for RSV-A-specific neutralizing antibodies (NT Abs).

### Secondary Objectives:

To assess the safety of RSV vaccine by the incidence of:
Solicited local reactions and systemic AEs up to Day 7;
Unsolicited AEs up to Day 28;
Moderate to severe LRTI (adverse event of special interest, AESI);
Clinical-safety laboratory parameters.

To assess the immunogenicity of RSV vaccine by evaluating up to Day 180:
RSV-A-specific and RSV-B-specific NT-Ab titers;
RSV prefusion F-protein-specific IgG titers;
The ratios of fold rises in RSV prefusion F-protein-specific IgG titers over fold rises in RSV-A-specific NT-Ab titers.

### Exploratory Objectives:

To further explore the immunogenicity of the RSV vaccine by assessing:
The epitope-specificity of IgGs to the RSV prefusion F-protein;
Core-VLP-specific IgG titers;
RSV prefusion F-protein-specific memory B-cell frequencies by enzyme-linked immune absorbent spot (ELISpot);
RSV prefusion F-protein-specific T-cell frequencies by ELISpot;
Human metapneumovirus (hMPV) NT-Ab titers at Days 0 and 28.

### Investigational Medicinal Product

### RSV Vaccine

Investigational vaccine was formulated at one concentration, 250 μg/0.5 mL as an aqueous vaccine or adsorbed to 500 μg aluminum hydroxide as an adjuvant. The lower dosage vaccines (25 and 75 μg) for each formulation were dilutions of the highest dose and were prepared just prior to administration using either aqueous or aluminum hydroxide adjuvant diluent. The aluminum hydroxide content was the same for all adjuvant formulations. All RSV vaccine formulations were administered as 0.5 mL doses.

### Placebo

Sterile aqueous diluent, delivered as a 0.5 mL dose. The placebo does not contain preservatives.

### Criteria for Evaluation and Analyses:

Co-Primary Endpoints (Safety): SAEs, MAAEs and AEs leading to study withdrawal from Day 0 up to the end of study.

Co-Primary Endpoints (Immunogenicity): Based on RSV-A-specific NT Abs at Day 28: SRR (percentage of subjects with ≥4-fold rise in titer versus baseline (Day 0)) and GMFR versus baseline (Day 0).

### Secondary Endpoints (Safety):

Solicited local reactions and systemic AEs from Day 0 to Day 7;
Unsolicited AEs from Day 0 to Day 28;
Moderate to severe LRTI from Day 0 up to the end of study;
Clinical-safety laboratory parameters at screening, and after dosing, at Days 0, 7 and 28.

### Secondary Endpoints (Immunogenicity):

Based on RSV-A-specific NT Abs and RSV-B-specific NT Abs: GMFR in titers versus baseline (Day 0; GMFR) at Days 7 and 180; SRRs to either RSV strain at Days 7 and 180; Percentages of subjects with ≥4-fold rise in titers versus baseline to either RSV strain at Days 7, 28 and 180; Percentages of subjects with ≥8-fold rise in titers versus baseline to either RSV strain at Days 7, 28 and 180; SRRs to both RSV strains at Days 7, 28 and 180; Geometric mean titer (GMT) to both RSV strains at Days 0, 7, 28 and 180.
Based on RSV prefusion F-protein-specific IgGs: GMFR at Days 7, 28 and 180.
Based on the ratio of fold rise versus baseline in RSV prefusion F-protein-specific IgG titer over fold rise versus baseline in RSV-A-specific NT-Ab titer: Geometric mean ratio (GMR) at Days 0, 7, 28 and 180.

### Exploratory Endpoints (Immunogenicity):

RSV prefusion F-protein-specific IgG titers, epitope-mapped by competitive binding in the presence of monoclonal antibodies at Days 0, 28, and 180;
Core-VLP-specific IgG titers at Days 0, 28, and 180;
RSV prefusion F-protein-specific memory-B-cell ELISpot frequencies at Days 0 and 7;
RSV prefusion F-protein-specific T-cell ELISpot frequencies at Days 0 and 7;
hMPV NT-Ab titers at Days 0 and 28.

### Example 6: Phase 1b Extension and Revaccination Study

This example describes a study to assess the immunogenicity out to 12 months from the single dose given in the study in Example 5 as well as the effect of revaccination with RSV vaccine.

RSV vaccine is administered to up to 120 older adults (e.g., adults of over 60 years of age) at a 75 μg unadjuvanted IVX-121 dose. These subjects may be a subset of the older adult study population described in Example 5 infra. Subjects are revaccinated 12 months after the initial dose and efficacy and safety are assessed six months after the revaccination dose. Safety and efficacy endpoints are those described in Example 5.

### Example 7: Interim Results of Phase 1/1b Study

This Example provides interim results from Example 5: a Phase 1/1b clinical trial of IVX-121, a VLP displaying a prefusion stabilized Respiratory Syncytial Virus (RSV) F antigen, in young and older adults. IVX-121 demonstrated a robust immunologic response in both young and older adult groups.

### IVX-121 Phase 1/1b Trial Design

The Phase 1/1b clinical trial of IVX-121 is a randomized, observer-blinded, placebo-controlled, multi-center study designed to evaluate the safety and immunogenicity of three dose levels of IVX-121, with and without aluminum hydroxide adjuvant, in healthy young and older adults. The study design is shown in FIG. 4.

The Phase 1 part of the trial enrolled 90 healthy young adults aged 18-45 years. The Phase 1b part of the trial enrolled 130 healthy older adults aged 60-75 years. Subjects were administered a single dose of IVX-121 at one of three dose levels (25, 75, 250 μg), with or without aluminum hydroxide adjuvant, or placebo.

The primary outcomes of the study were safety and immunogenicity up to 28 days post-vaccination; neutralizing antibodies to RSV-A and RSV-B were measured in international units (IU/mL) using the WHO international reference standard.

### Topline Results

### Safety

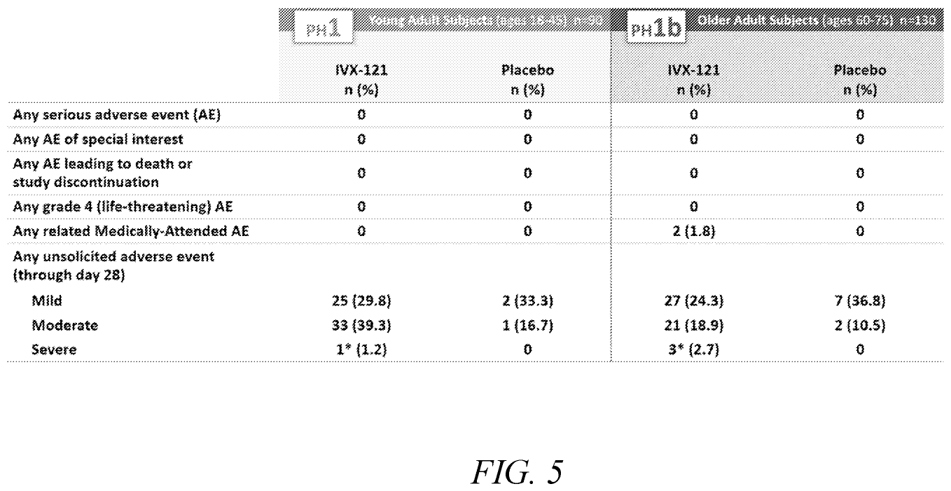

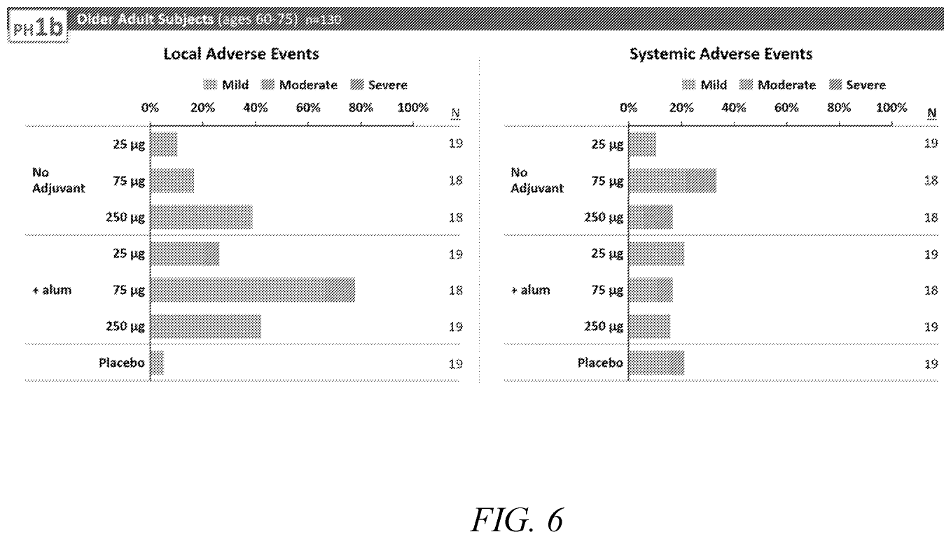

In this Phase 1/1b study, IVX-121 was generally well-tolerated across all dosage groups. Solicited local and systemic adverse events (AEs) were generally mild or moderate, without dose-limiting reactogenicity. In the older adult target population, across the six dosage groups for IVX-121 with or without adjuvant, the proportion of subjects experiencing any systemic AE within seven days was 11-33%, and similar to 21% for placebo. The most common local and systemic AEs were injection site tenderness, headache and fatigue. There were no serious AEs related to vaccine, AEs of special interest, or AEs leading to discontinuation. Data are shown in FIG. 5 and FIG. 6.

### Immunogenicity:

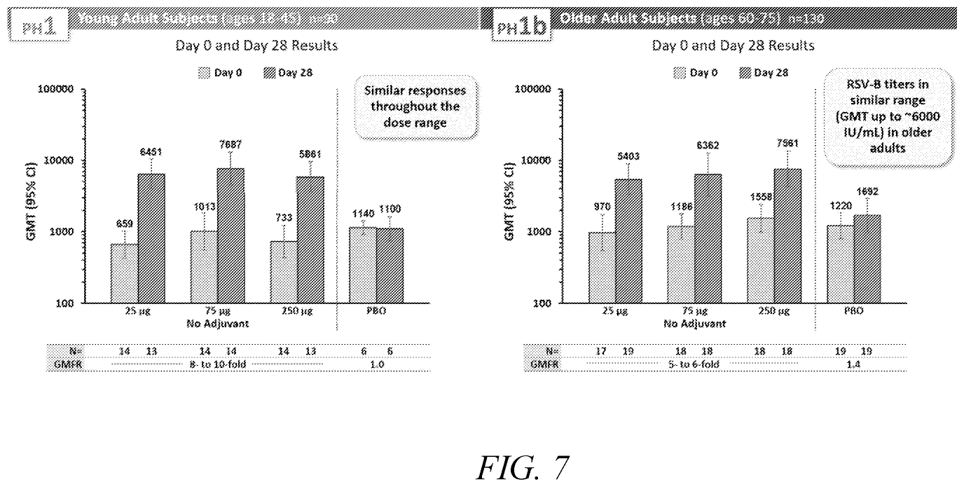

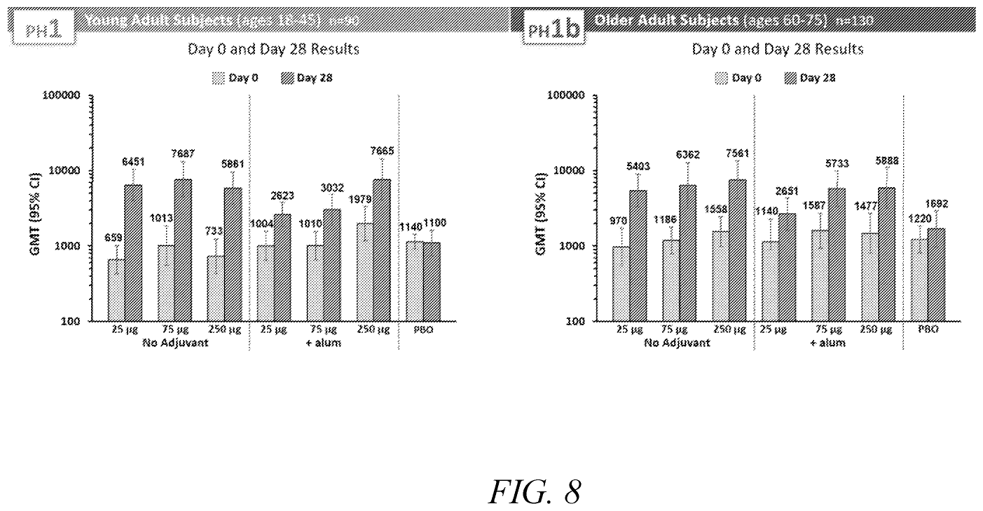

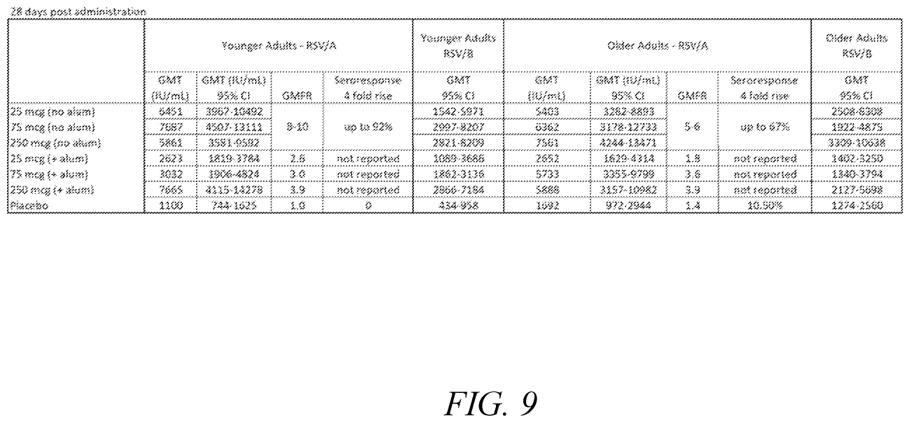

IVX-121 induced a robust immune response in both young and older adult groups. Data indicated a dose-independent response, including at the lowest non-adjuvanted dose (25 μg) (FIG. 7). No additional benefit from the aluminum hydroxide adjuvant was observed at any dosage level in either portion of the study (FIG. 8). Geometric mean titers for RSV-A and RSV-B were in comparable ranges for both groups (FIG. 9)

### Young Adults (Phase 1):

In young adults, across dosage groups, IVX-121 induced Geometric Mean Titers (GMT) in RSV-A neutralizing antibodies (nAbs) of up to 7,687 IU/mL compared to 1,100 IU/mL for placebo at Day 28. These titers corresponded to a Geometric Mean Fold Rise (GMFR) versus baseline up to 10-fold for IVX-121 at Day 28.

### Older Adults (Phase 1b):

GMT responses in IU/mL for older adults were comparable with those for young adults in the Phase 1 portion of this study. Across dosage groups, IVX-121 induced GMT in RSV-A nAbs of up to 7,561 IU/mL compared to 1,692 IU/mL for placebo at Day 28. GMFR at Day 28 was up to 6-fold, reflecting higher baseline titers in the older adults group.

### Conclusion

These data indicate that IVX-121 was generally well tolerated and elicited a strong and consistent response against RSV in healthy young and older adults. These data are particularly encouraging for the vulnerable older adult population with co-morbidities and increased risk for severe disease and hospitalization. IVX-121 exhibits immunogenicity at very low microgram dosage levels and tolerability to the highest dose level. Eligible older adults from the Phase 1b cohort will be followed out to 12 months to assess durability of response.

### Neutralizing Antibody Assay Methods

Validated RSV/A and RSV/B neutralizing antibody (NAb) assays were used to measure the presence of RSV-specific neutralizing antibodies. These assays were been validated for standard bioanalytical assay parameters to include Specificity, Accuracy, Precision, Repeatability (intra-assay precision), Intermediate Precision, Linearity, Dilutional Linearity, Range, Stability, and Robustness. Critical reagents for these assays are commercially purchased and qualified for use. In brief, human serum samples are serially diluted and incubated with a constant concentration of virus (RSV A2 (ATCC, Cat #VR-1540) and RSV B 18537 (ATCC, Manassas, VA; Cat #VR-1580)). Neutralization is measured by the inhibition of virus propagation in HEp-2 cells (ATCC, Cat #CCL23). Following an incubation period, cells are fixed and immunostained with a murine monoclonal antibody directed against RSV F protein, followed by horse radish peroxidase (HRP)-conjugated goat-anti-mouse antibody and TrueBlue (TB). The plates are scanned with an UV Analyzer and Spot counts (SC; or spot forming cells, SCF) per well at each serum/antibody concentration are quantified. The values are then analyzed to determine the dilution of serum/antibody that yield a selected reduction point (i.e., 50% or IC50).

Since there are different assay formats available for RSV neutralization assays, it is important to have a reference antiserum to standardize results. The first International Standard for antiserum to RSV/A and RSV/B (NIBSC code: 16/284) was established by the WHO Expert Committee on Biological Standardization in 2017 (McDonald et al., 2017; McDonald et al., 2018; McDonald et al., 2020). This Reference Standard was shown to be suitable for the standardization of virus neutralization methods to measure antibody levels against RSV/A and RSV/B in human sera. Due to limited quantities available of the Reference Standard, bioanalytical labs are encouraged to generate an in-house reference (IHR) standard by calibrating the IHR to the International Reference Standard. The RSV Reference Standard (16/284) with an assigned a potency of 1000 International Units (IU) anti-RSV/A or anti-RSV/B neutralizing antibodies per ampoule was used to establish and validate an IHR to be included during neutralization titer analyses. The potency of the IHR standard was determined to be 1,831 IU/mL for RSV/A and 609 IU/mL for RSV/B. These potencies were used to calculate conversion factors which can be used for converting MN titer results (AU/mL) to IU/mL. The potency of a test sample in IU/mL was calculated by multiplying the test sample MN titer by 1.0578 for RSV/A and by 0.6936 for RSV/B. The RSV/A and RSV/B NAb assays has been validated to an assay range of 9.4-180263 AU/mL and 8-195392 AU/mL, respectively. The validated assay range in IU/mL for RSV/A and RSV/B are 9.9-190682 and 5.5-135524, respectively.

McDonald et al. 2017. Report on the WHO collaborative study to establish the 1st international standard for antiserum to respiratory syncytial virus (No. WHO/BS/2017.2318). World Health Organization. https://apps.who.int/iris/handle/10665/260488

McDonald et al. 2018. Establishment of the first WHO International Standard for antiserum to Respiratory Syncytial Virus: Report of an international collaborative study. Vaccine, 36, 7641-7649.

McDonald et al. 2020. Expansion of the 1st WHO international standard for antiserum to respiratory syncytial virus to include neutralisation titres against RSV subtype B: An international collaborative study. Vaccine, 38, 800-807.

WHO 2020. WHO International Standard 1st International Standard for Antiserum to Respiratory Syncytial Virus NIBSC code: 16/284 Instructions for use (Version 4.0, Dated Jan. 4, 2020). https://www.nibsc.org/documents/ifu/16-284.pdf

### Example 8: Prophetic Phase 2 or Phase 3 Study

This Example prophetically describes a Phase 2 or Phase 3 study of IVX-121. IVX-121 is formulated as a non-adjuvanted vaccine. The vaccine is administered in a single dose of at most about 25 μg, such as 1 μg, 2.5 μg, 5 μg, 7.5 μg, 10 μg, 12.5 μg, 15 μg, 17.5 μg, 20 μg, 22.5 μg, or 25 μg of the IVX-121 protein complex. Subjects may be selected as adults 18+ years of age or adults 60+ years of age, or as individuals at high risk of severe disease (e.g., affected by underlying chronic conditions, including diabetes mellitus, cardiovascular diseases, and respiratory diseases; frail elderly; or immunocompromised). Clinical endpoints include RSV-associated LRTD (lower respiratory tract disease), moderate/severe RSV-associated LRTD, and/or RSV-associated acute respiratory disease. Subjects are monitored for one, two, or three RSV seasons. The vaccine may be administered again after three to five years.

In addition, human challenge studies may be performed in adults aged 18-50 challenged with RSV/A virus four weeks after receiving as single dose of the vaccine. Clinical endpoints may include polymerase chain reaction (PCR)-confirmed RSV infection and self-reported symptoms.

In each study, efficacy is observed at doses at or below 75 μg of the IVX-121 protein complex and/or at or below 25 μg of the IVX-121 protein complex.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A pharmaceutical composition, comprising a protein complex comprising a first component comprising an RSV F protein and a first multimerization domain; and one or more pharmaceutically acceptable diluents or excipients.

2. The pharmaceutical composition of claim 1, wherein the protein complex comprises 2, 3, 4, 5 or more copies of the first component.

3. The pharmaceutical composition of claim 1 or claim 2, wherein the protein complex comprises a second component comprising a second multimerization domain.

4. The pharmaceutical composition of claim 3, wherein the protein complex comprises 2, 3, 4, 5 or more copies of the second component.

5. The pharmaceutical composition of any one of claims 1 to 4, wherein the protein complex comprises a third component comprising a third multimerization domain.

6. The pharmaceutical composition of claim 5, wherein the protein complex comprises 2, 3, 4, 5 or more copies of the third component.

7. The pharmaceutical composition of any one of claims 1 to 6, wherein the protein complex is a nanostructure, nanoparticle, or protein-based virus-like particle.

8. The pharmaceutical composition of any one of claims 1 to 7, wherein the components of the protein complex are arranged according to a set of symmetry operators forming dihedral symmetry group.

9. The pharmaceutical composition of any one of claims 1 to 8, wherein the components of the protein complex are arranged according to a set of symmetry operators forming cyclic symmetry group.

10. The pharmaceutical composition of any one of claims 1 to 9, wherein the protein complex is an icosahedral protein complex.

11. The pharmaceutical composition of any one of claims 1 to 9, wherein the protein complex is a tetrahedral protein complex.

12. The pharmaceutical composition of any one of claims 1 to 9, wherein the protein complex is an octahedral protein complex.

13. The pharmaceutical composition of claims 1 to 12, wherein the first multimerization domain is a trimerization domain and/or the second multimerization domain is a pentamerization domain.

14. The pharmaceutical composition of any one of claims 2 to 13, wherein the protein complex comprises 20 copies of the first component and 12 copies of the second component.

15. The pharmaceutical compositions of any one of claims 1 to 14, wherein the RSV F protein comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 14, 34, and 35.

16. The pharmaceutical compositions of any one of claims 1 to 15, wherein the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 24 and 30-31; and/or wherein the second multimerization domain comprises an amino acid sequence which is at least 95%, at least 96%, at least 97%, at least 98%, at least 99%, or at least 100% identical to an amino acid sequence selected from any one of SEQ ID NOS: 22-23, 25-29, and 32.

17. The pharmaceutical composition of any one of claims 1 to 16, wherein the first component comprises an amino acid sequence which is at least 75%, 80%, 85%, 90%, 91%, 92%, 93%, 94%, 95%, 96%, 97%, 98%, 99%, or 100% identical to SEQ ID NO: 6; and
wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 26.

18. The pharmaceutical composition of any one of claims 1 to 17, wherein the pharmaceutical composition comprises an oil-in-water adjuvant.

19. The pharmaceutical composition of any one of claims 1 to 17, wherein the pharmaceutical composition comprises an aluminum hydroxide-adjuvant.

20. A unit dose of the pharmaceutical composition of any one of claims 1 to 19, wherein the unit dose comprises between about 0.5 μg and about 500 μg of the protein complex.

21. The unit dose of claim 20, wherein the unit dose comprises between about 25 μg and about 250 μg of the protein complex.

22. The unit dose of claim 20, wherein the unit dose comprises between about 25 μg and about 75 μg of the protein complex.

23. The unit dose of claim 20, wherein the unit dose comprises between about 75 μg and about 250 μg of the protein complex.

24. The unit dose of claim 20, wherein the unit dose comprises about 25 μg, about 75 μg, or about 250 μg of the protein complex.

25. The unit dose of claim 20, wherein the unit dose comprises at most about 25 μg, at most about 75 μg, or at most about 250 μg of the protein complex.

26. The unit dose of claim 20, wherein the unit dose comprises at most 1 μg, at most 2.5 μg, at most 5 μg, at most 7.5 μg, at most 10 μg, at most 12.5 μg, at most 15 μg, at most 17.5 μg, at most 20 μg, at most 22.5 μg, or at most 25 μg of the protein complex.

27. The unit dose of claim 20, wherein the unit dose comprises 1 μg, 2.5 μg, 5 μg, 7.5 μg, 10 μg, 12.5 μg, 15 μg, 17.5 μg, 20 μg, 22.5 μg, or 25 μg of the protein complex.

28. A method of vaccinating a subject, comprising administering to the subject an effective amount of the pharmaceutical composition of any one of claims 1 to 19 or the unit dose of any one of claims 20 to 27.

29. A method of generating an immune response in a subject, comprising administering to the subject an effective amount of the pharmaceutical composition of any one of claims 1 to 19 or the unit dose of any one of claims 20 to 27.

30. The method of claim 29, wherein the method concurrently generates an immune response to a human metapneumovirus (hMPV) F protein through cross-reactivity with the RSV F protein.

31. A method of treating and/or preventing severe lower respiratory tract infection (LRTI) associated caused by RSV in a subject, comprising administering to the subject an effective amount of the pharmaceutical composition of any one of claims 1 to 19 or the unit dose of any one of claims 20 to 27.

32. A method of preventing RSV disease in a subject, comprising administering to the subject an effective amount of the pharmaceutical composition of any one of claims 1 to 19 or the unit dose of any one of claims 20 to 27.

33. The method of any one of claims 28 to 32, wherein the subject is at risk of severe RSV disease.

34. The method of claim 33, wherein the subject is at risk of severe RSV disease because of underlying diabetes mellitus, cardiovascular disease, or respiratory disease.

35. The method of any one of claims 28 to 34, wherein the subject is an adult of over 50 years of age, an adult of over 55 years of age, or an adult of over 60 years of age.

36. The method of any one of claims 28 to 34, wherein the subject is an adult at least 50 years of age, an adult at least 55 years of age, or an adult at least 60 years of age.

37. The method of any one of claims 28 to 34, wherein the subject is an adult of 18-45 years of age.

38. The method of any one of claims 28 to 34, wherein the subject is a healthy adult of 18-45 years of age.

39. The method of any one of claims 28 to 34, wherein the subject is over 18 years of age.

40. The method of any one of claims 28 to 34, wherein the subject is 18 years of age or greater.

41. The method of any one of claims 28 to 34, wherein the subject is 18 years of age or less.

42. A method of generating an immune response in an unborn child, the method comprising administering an effective amount of the pharmaceutical composition of any one of claims 1 to 19 or the unit dose of any one of claims 20 to 27 to the mother of said unborn child.

43. The method of claim 42, wherein the pharmaceutical composition is administered to the mother in the last trimester of the pregnancy.

44. A method of generating an immune response in an infant and/or prevent respiratory syncytial virus (RSV) disease in an infant through maternal immunization of a pregnant subject, the method comprising administering an effective amount of the pharmaceutical composition of any one of claims 1 to 19 or the unit dose of any one of claims 20 to 27 to the subject.

45. The method of any one of claims 28 to 44, wherein the effective amount is between about 0.5 μg and about 500 μg of the protein complex.

46. The method of claim 45, wherein the effective amount is between about 25 μg and about 250 μg of the protein complex.

47. The method of claim 45, wherein the effective amount is between about 25 μg and about 75 μg of the protein complex.

48. The method of claim 45, wherein the effective amount is between about 75 μg and about 250 μg of the protein complex.

49. The method of claim 45, wherein the effective amount is about 25 μg, about 75 μg, or about 250 μg of the protein complex.

50. The method of claim 45, wherein the effective amount is at most about 25 μg, at most about 75 μg, or at most about 250 μg of the protein complex.

51. The method of claim 45, wherein the unit dose comprises at most 1 μg, at most 2.5 μg, at most 5 μg, at most 7.5 μg, at most 10 μg, at most 12.5 μg, at most 15 μg, at most 17.5 μg, at most 20 μg, at most 22.5 μg, or at most 25 μg of the protein complex.

52. The method of claim 45, wherein the unit dose comprises 1 μg, 2.5 μg, 5 μg, 7.5 μg, 10 μg, 12.5 μg, 15 μg, 17.5 μg, 20 μg, 22.5 μg, or 25 μg of the protein complex.

53. The method of any one of claims 28 to 52, further comprising administering a second dose of the pharmaceutical composition.

54. The method of claim 53, wherein the second dose is administered within about 2, 3, 4, 5, 6, 7, 8, 9, 10, 11, or 12 weeks, about 1 month, about 2 months, about 3 months, about 4 months, about 5 months, about 6 months, about 9 months, about 12 months, about 24 months, or about 36 months of the first dose.

55. The method of claim 53 or claim 54, further comprising administering a third dose of the pharmaceutical composition.

56. The method of claim 55, wherein the third dose is administered about 1 year, about 2 years, about 3 years, about 4 years, about 5 years, about 6 years, about 7 years, about 8 years, about 9 years, or about 10 years after the second dose.

57. The method of claim 55 or claim 56, further comprising administering subsequent doses at regular intervals of about 1, 2, 3, 4 5, 6, 7, 8, 9, or 10 years.

58. The method of any one of claims 28 to 57, wherein the method limits the development of an RSV infection in a subject.

59. The method of any one of claims 28 to 57, wherein the method limits the development of more severe lower respiratory tract infection (LRTI) in the subject.

60. The method of any one of claims 28 to 57, wherein the method results in the production of RSV-A-specific neutralizing antibodies in the subject.

61. The method of claim 60, wherein the method results in an increase in RSV-A-specific neutralizing antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

62. The method of claim 60 or claim 61, wherein the increase in RSV-A-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

63. The method of any one of claims 28 to 62, wherein the method results in the production of RSV-B-specific neutralizing antibodies in the subject.

64. The method of claim 63, wherein the method results in an increase in RSV-B-specific neutralizing antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

65. The method of claim 63 or claim 64, wherein the increase in RSV-B-specific neutralizing antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

66. The method of any one of claims 28 to 65, wherein the method results in the production of RSV F-protein-specific IgG antibodies in the subject.

67. The method of claim 66, wherein the method results in an increase of RSV F-protein-specific IgG antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

68. The method of claim 66 or claim 67, wherein the increase in RSV F-protein-specific IgG antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

69. The method of any one of claims 58 to 68, wherein the method results in the production of Core-VLP-specific IgG antibodies in the subject.

70. The method of claim 69, wherein the method results in an increase in Core-VLP-specific IgG antibodies in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

71. The method of claim 69 or claim 70, wherein the increase in Core-VLP-specific IgG antibodies is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

72. The method of any one of claims 58 to 68, wherein the method results in the production of substantially no Core-VLP-specific IgG antibodies in the subject.

73. The method of any one of claims 58 to 72, wherein the method results in the production of RSV F-protein-specific memory-B-cells in the subject.

74. The method of claim 73, wherein the method results in an increase in RSV F-protein-specific memory-B-cells in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

75. The method of claim 73 or claim 74, wherein the increase in RSV F-protein-specific memory-B-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

76. The method of any one of claims 58 to 75, wherein the method results in the production of RSV F-protein-specific T-cells in the subject.

77. The method of claim 76, wherein the method results in an increase in RSV F-protein-specific T-cells in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

78. The method of claim 76 or claim 77, wherein the increase in RSV F-protein-specific T-cells is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

79. The method of any one of claims 58 to 78, wherein the method results in the production of neutralizing antibodies against human metapneumovirus in the subject.

80. The method of claim 79, wherein the method results in an increase in neutralizing antibodies against human metapneumovirus in the subject of at least about 2-fold, at least about 3-fold, at least about 4-fold, at least about 5-fold, at least about 8-fold, at least about 10-fold, at least about 15-fold, at least about 20-fold, or at least about 25-fold compared to baseline.

81. The method of claim 79 or claim 80, wherein the increase in antibodies against human metapneumovirus is detectable within about one week, within about 2 weeks, within about 3 weeks, within about 4 weeks, within about 5 weeks, within about 6 weeks, within about 7 weeks, within about 8 weeks, within about 9 weeks, within about 10 weeks, within about 11 weeks, or within about 12 weeks of administration of the pharmaceutical composition.

82. The method of any one of claims 28 to 81, wherein the method prevents severe LRTI associated with or caused by RSV more effectively than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

83. The method of any one of claims 28 to 82, wherein the method prevents severe LRTI associated with or caused by RSV more effectively than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

84. The method of any one of claims 28 to 83, wherein the method results in greater RSV-A and/or RSV-B-specific neutralizing antibodies than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

85. The method of any one of claims 28 to 84, wherein the method results in protective immunity for a longer time period than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

86. The method of any one of claims 28 to 85, wherein the method results in a neutralizing antibody response lasting at least 12 months.

87. The method of any one of claims 28 to 86, wherein the method results in a protective immunity lasting at least 12 months.

88. The method of any one of claims 28 to 87, wherein the adjuvant, if present, increases the durability of the neutralizing antibody response, the cross-protection of the neutralizing antibody response, and/or the magnitude of the B cell or T cell activation in the subject.

89. The method of any one of claims 28 to 88, wherein the method causes fewer adverse events than a method involving administration of a trimeric antigen having an amino acid sequence which is at least 75%, 80%, 85%, 90%, 91%, 92%, 93%, 94%, 95%, 96%, 97%, 98%, 99%, or 100% identical to an amino acid sequence of any one of SEQ ID NOs: 7, 14, 34, and 35.

90. The method of any one of claims 28 to 89, wherein the method results in RSV-A and/or RSV-B specific neutralizing antibodies in the subject at geometric mean titer (GMT) of greater than 1,000 international units per milliliter (IU/mL), greater than 2,000 IU/mL, greater than 3,000 IU/mL, greater than 4,000 IU/mL, greater than 5,000 IU/mL, greater than 6,000 IU/mL, greater than 7,000 IU/mL, greater than 8,000 IU/mL, greater than 9,000 IU/mL, or greater than 10,000 IU/mL.

91. The method of any one of claims 28 to 89, wherein the method results in RSV-A and/or RSV-B specific neutralizing antibodies in the subject at GMT of greater than 1,000 IU/mL, greater than 2,000 IU/mL, greater than 3,000 IU/mL, greater than 4,000 IU/mL, greater than 5,000 IU/mL, greater than 6,000 IU/mL, greater than 7,000 IU/mL, greater than 8,000 IU/mL, greater than 9,000 IU/mL, or greater than 10,000 IU/mL, wherein the subject is an adult of age 18 to 45.

92. The method of any one of claims 28 to 89, wherein the method results in RSV-A and/or RSV-B specific neutralizing antibodies in the subject at GMT of greater than 1,000 IU/mL, greater than 2,000 IU/mL, greater than 3,000 IU/mL, greater than 4,000 IU/mL, greater than 5,000 IU/mL, greater than 6,000 IU/mL, greater than 7,000 IU/mL, greater than 8,000 IU/mL, greater than 9,000 IU/mL, or greater than 10,000 IU/mL, wherein the subject is an adult of age 60 to 75.

93. The method of any one of claims 28 to 89, wherein the method results in RSV-A specific neutralizing antibodies in the subject at GMT of greater than 3,000 IU/mL.

94. The method of any one of claims 28 to 89, wherein the method results in RSV-A specific neutralizing antibodies in the subject at GMT of greater than 3,000 IU/mL, wherein the subject is an adult of age 18 to 45.

95. The method of any one of claims 28 to 89, wherein the method results in RSV-A specific neutralizing antibodies in the subject at GMT of greater than 3,000 IU/mL, wherein the subject is an adult of age 60 to 75.

96. The method of any one of claims 28 to 95, wherein the method results in prefusion RSV F binding IgG antibodies in the subject at GMT of greater than 1,000 immunosorbent assay unit per milliliter (EU/mL), greater than 2,000 EU/mL, greater than 3,000 EU/mL, greater than 4,000 EU/mL, greater than 5,000 EU/mL, greater than 6,000 EU/mL, greater than 7,000 EU/mL, greater than 8,000 EU/mL, greater than 9,000 EU/mL, or greater than 10,000 EU/mL.

97. The method of any one of claims 28 to 96, wherein the method results in prefusion RSV F binding IgG antibodies in the subject at GMT of greater than 1,000 EU/mL, greater than 2,000 EU/mL, greater than 3,000 EU/mL, greater than 4,000 EU/mL, greater than 5,000 EU/mL, greater than 6,000 EU/mL, greater than 7,000 EU/mL, greater than 8,000 EU/mL, greater than 9,000 EU/mL, or greater than 10,000 EU/mL, wherein the subject is an adult of age 18 to 45.

98. The method of any one of claims 28 to 97, wherein the method results in prefusion RSV F binding IgG antibodies in the subject at GMT of greater than 1,000 EU/mL, greater than 2,000 EU/mL, greater than 3,000 EU/mL, greater than 4,000 EU/mL, greater than 5,000 EU/mL, greater than 6,000 EU/mL, greater than 7,000 EU/mL, greater than 8,000 EU/mL, greater than 9,000 EU/mL, or greater than 10,000 EU/mL, wherein the subject is an adult of age 60 to 75.

99. The method of any one of claims 28 to 98, wherein the method results in RSV-A specific neutralizing antibodies in the subject at GMT of greater than 3,000 EU/mL.

100. The method of any one of claims 28 to 99, wherein the method results in RSV-A specific neutralizing antibodies in the subject at GMT of greater than 3,000 EU/mL, wherein the subject is an adult of age 18 to 45.

101. The method of any one of claims 28 to 100, wherein the method results in RSV-A specific neutralizing antibodies in the subject at GMT of greater than 3,000 EU/mL, wherein the subject is an adult of age 60 to 75.

102. The method of any one of claim 28 to 101, wherein the method results in an RSV-A seroresponse rate at 4-fold rise or 8-fold rise of at least 20%, at least 30%, at least 40%, at least 50%, at least 60%, at least 70%, at least 80% or at least 90%.

103. The method of any one of claim 28 to 102, wherein the method results in an RSV-A seroresponse rate at 4-fold rise or 8-fold rise of 20% to 40%, 30% to 50%, 40% to 60%, 50% to 70%, 60% to 80%, or 70% to 90%.

104. The method of any one of claim 28 to 103, wherein the method results in an RSV-B seroresponse rate at 4-fold rise or 8-fold rise of at least 20%, at least 30%, at least 40%, at least 50%, at least 60%, at least 70%, at least 80% or at least 90%.

105. The method of any one of claim 28 to 104, wherein the method results in an RSV-B seroresponse rate at 4-fold rise or 8-fold rise of 20% to 40%, 30% to 50%, 40% to 60%, 50% to 70%, 60% to 80%, or 70% to 90%.

106. The method of any one of claims 28 to 105, wherein the method results in serious adverse events in fewer than 20%, fewer than 10%, fewer than 5%, or about 0% of subjects administered the pharmaceutical composition.

107. The method of any one of claims 28 to 105, wherein the method results in serious adverse events in fewer than 20%, fewer than 10%, fewer than 5%, or about 0% of subjects administered the pharmaceutical composition, wherein the subjects are adults of age 18 to 45.

108. The method of any one of claims 28 to 105, wherein the method results in serious adverse events in fewer than 20%, fewer than 10%, fewer than 5%, or about 0% of subjects administered the pharmaceutical composition, wherein the subjects are adults of age 60 to 75.

109. The method of any one of claims 28 to 105, wherein the method results in serious adverse events in fewer than 20%, fewer than 10%, fewer than 5%, or about 0% of subjects administered the pharmaceutical composition.

110. The method of any one of claims 28 to 105, wherein the method results in serious adverse events in fewer than 20%, fewer than 10%, fewer than 5%, or about 0% of subjects administered the pharmaceutical composition, wherein the subjects are adults of age 18 to 45.

111. The method of any one of claims 28 to 105, wherein the method results in serious adverse events in fewer than 20%, fewer than 10%, fewer than 5%, or about 0% of subjects administered the pharmaceutical composition, wherein the subjects are adults of age 60 to 75.

112. The method of any one of claims 28 to 111, wherein the pharmaceutical composition is substantially free of any adjuvant.

113. The method of any one of claims 28 to 112, wherein the pharmaceutical composition is substantially free of aluminum salt adjuvants.

114. The method of any one of claims 28 to 112, wherein the pharmaceutical composition is substantially free of alum.
